(19) United States
(12) Patent Application Publication
(10) Pub. No.: US 2010/0048923 A1
(43) Pub. Date: Feb. 25, 2010

(54) ATR Inhibitor

(76) Inventors: Hiroshi Nishida, Niigata (JP); Yasuo Hamamori, Houston, TX (US); Tetsuya Konishi, Niigata (JP)

Correspondence Address: FROMMER LAWRENCE & HAUG, 745 FIFTH AVENUE- 10TH FL., NEW YORK, NY, 10151, US

(21) Appl. No.: 12/090,898

(22) PCT Filed: Oct. 18, 2006
(86) PCT No.: PCT/JP2006/320756

§ 371 Date: Apr. 21, 2008

(30) Foreign Application Priority Data
Oct. 21, 2005 (JP) 2005-307534

(51) Int. Cl. C07D 317/70; C07C 43/23; C07D 493/04

(52) U.S. Cl. 549/432; 568/633

(57) ABSTRACT

The present invention is to provide ATR inhibitor containing tricyclic compound including Schizandrins and Gomisins as an active constituent, useful for an inhibitor of ATR protein kinase.

### FIELD OF INVENTION

The present invention relates to an ATR inhibitor to inhibit the activity of ATR (ataxia-telangiectasia mutated and rad3-related), more particularly, an ATR inhibitor of a tricyclic compound as an active constituent.

### RELATED ART

It is known that ATM (ataxia-telangiectasia mutated) and ATR (ataxia-telangiectasia mutated and rad3-related) protein kinase (hereinafter, referred also to as “ATM” and “ATR”) are playing a crucial role in cellular DNA damage (Non-Patent-Related Document 1). Ultra violet (UV) radiation and ionizing radiation (IR) induce DNA damage and exert the checkpoint signal cascades followed by cell cycle arrest as G1/S, intra-S and G2/M checkpoint in order to repair the DNA damage, or squeeze the cells into apoptosis. The cells derived from AT patient, ATM deficient, are hypersensitive to IR because it exhibits the phenotype of G1/S and G2/M checkpoint abrogation. Therefore, ATM and its downstream pathways are important for the maintenance of chromatin. Moreover, transient or stable deficiency of ATR causes to prominent instability of chromosome or early embryonic lethal (Non-Patent-Related Document 2). Interestingly, it is known that the phosphorylation of p53 protein (hereinafter, referred to as “p53”) or NBS1 with ATR delays in the absence of ATM. It suggests that ATR is a cooperative partner of ATM in terms of DNA damage response. Indeed, ATR is playing a crucial role for protecting from DNA damage. These are also indicating that the regulation of ATM or ATR protein activities is not only required for the maintenance of genome stability, but also applicable for anti-cancer therapy such as chemotherapy and radiation therapy.

Non-Patent-Related Document 3 discloses that caffeine (3,7-dihydro-1,3,7-trimethyl-1H-purine-2,6-dione)-assisted chemotherapy minimizes tumber excision for non-metastatic osteosarcoma. It is perceived that caffeine inhibits ATM and ATR protein kinase activities in the response of DNA damage induced by chemotherapy. It is also known that caffeine inhibits the activity of ATM rather than that of ATR.

Critical roles of ATR and ATM have been investigated in recent studies such as the transfection siRNA into the cell or overexpression of a protein lacking the kinase active site of the recombinant kinase in the cell (Non-Patent-Related Documents 4 to 6). However, the less are reported the function of ATR in comparison with that of ATM because the stable knockout mice of ATR are lethal. In addition, in the current situation of specific inhibitors for ATR being unknown, the inhibition of ATM and ATR protein kinase activity by chemicals are widely investigated by using caffeine and wortmannin, an inhibitor of PI3 kinase. Therefore, the discovery of specific inhibitor for ATR will lead to biochemical knowledge in the DNA damage response and to many advantages to the clinical study of anti-cancer therapy. Therefore, it is desired to obtain compounds to inhibit ATR activities.

Among those, ATM and ATR phosphorylate p53 at Serine 15 during the response to the DNA damage in the cells. p53 plays a pivotal role in the G1/S checkpoint induced by DNA damage. Recent findings indicate that the serine at 15 of p53 is directly phosphorylated by ATR or ATM to arrest the cell cycle at G1 phase during the exposure of UV light. Thus, the phosphorylation of serine 15 in p53 is the reasonable index to estimate G1/S checkpoint function originated from ATM or ATR after DNA damage is induced by UV. In addition, such a phosphorylation reflects ATR activity in the damage response toward UV. As well as G1/S checkpoint, G2/M checkpoint is regulated by both ATR and ATM in DNA damaged cells. G2/M checkpoint is very important and well conserved in most of cancer cells either in p53 deficient or mutated tumors. Notably, the cells lacking p53 function were also sensitive to DNA damage by the abrogation of G2/M checkpoint. Indeed, ATR related G2/M checkpoint is essential for genome maintenance of proliferating cells. These results suggest that inhibition of the whole bunch related G2/M checkpoint wherein such an inhibition includes the inhibition of ATR protein kinase activity will be a candidate for the anti-cancer therapy.

On the other hand, Schizandrins and Gomisins are dibenzocyclooctadine derivatives derived from Schisandrae Fructus, and are compounds commonly used as Chinese medicine. As one of efficacy of these compounds, Non-Patent-Related Document 8 discloses that Schizandrin B induces caspase-3 dependent apoptosis in SMMC-7221 hepatoma cells. Now, other signal modulation or the other effects on various types of cells including tumors by this compound are remaining for further study. It is naturally believed that there are many possibilities in Schisandrae Fructus traditional Chinese medicine, including Schizandrins and Gomisins, based on the thousands year experiences. Over 70% of anti-cancer drugs are derived from natural products or its mimics with expectation of fewer side effects. We sought to apply Schizandrins and Gomisins on the signal transduction pathway of DNA damage response in terms of sensitizer of anti-cancer therapy.

### Patent Document 1

Japanese Patent Application Publication No. 206751/1995

### Patent Document 2

Japanese Patent Application Publication No. 192133/1994

### Patent Document 3

Japanese Patent Application Publication No. 048592/1990

### Patent Document 4

Japanese Patent Application Publication No. 123184/1993

### Patent Document 5

US Application Publication No. 2005/0282910

### Non-Patent-Related Document 1

Shiloh Y. et al., “ATM and ATR: networking cellular responses to DNA damage”, Curr. Opin. Genet. Dev., 2001, vol. 11, p. 71-7

### Non-Patent-Related Document 2

Cortez D. et al., “ATR and ATRIP: partners in checkpoint signaling”, Science, 2001, vol. 294, p. 1713-6

### Non-Patent-Related Document 3

Tsuchiya H. et al., “Caffeine-assisted chemotherapy and minimized tumor excision for nonmetastatic osteosarcoma.”, Anticancer Res., 1998, vol. 18, p. 657-66

### Non-Patent-Related Document 4

Shackelford R. E. et al., “The Ataxia telangiectasia gene product is required for oxidative stress-induced G1 and G2 checkpoint function in human fibroblasts.”, J. Biol. Chem., 2001, vol. 276, p. 21951-9

### Non-Patent-Related Document 5

Wright J. A. et al., “Protein kinase mutants of human ATR increase sensitivity to UV and ionizing radiation and abrogate cell cycle checkpoint control.”, Proc. Natl. Acad. Sci. USA., 1998, vol. 95, p. 7445-50

### Non-Patent-Related Document 6

Shigeta T. et al., “Defective control of apoptosis and mitotic spindle checkpoint in heterozygous carriers of ATM mutations.”, Cancer Res., 1999, vol. 59, p. 2602-7

### Non-Patent-Related Document 7

Sarkaria J. N. et al., “Inhibition of ATM and ATR kinase activities by the radiosensitizing agent, caffeine.”, Cancer Res., 1999, vol. 59, p. 4375-82

### Non-Patent-Related Document 8

Wu Y. F. et al., “Down-modulation of heat shock protein 70 and up-modulation of Caspase-3 during schisandrin B-induced apoptosis in human hepatoma SMMC-7221 cells”, World J. Gastroenterol., 2004, vol. 10, p. 2944-48

### Non-Patent-Related Document 9

Tibbetts et al., Gene & Development, 2000, vol. 14, p. 2989-3002

### Non-Patent-Related Document 10

Canman et al., Science, 1998, vol. 281, p. 1677-9

### Non-Patent-Related Document 11

Rouet et al., Proc. Natl. Acad. Sci. USA, 1994, vol. 91, p. 6064-8

### Non-Patent-Related Document 12

Chem. Pharm. Bull., 2003, vol. 51, No. 11, p. 1233-1236

### Non-Patent-Related Document 13

Acta Pharmacologica Sinica, 1998, vol. 19, No. 4, p. 313-316

### DISCLOSURE OF INVENTION

### Problems to be Solved by the Invention

Hence, the present invention is to provide ATR inhibitor containing tricyclic compound including Schizandrins and Gomisins as an active constituent, useful for an inhibitor of ATR protein kinase.

### Means to Solve the Problems

The ATR inhibitor according to the present invention is characterized in that:

ATR inhibitor containing a compound having a following formula (1) as an active constituent.

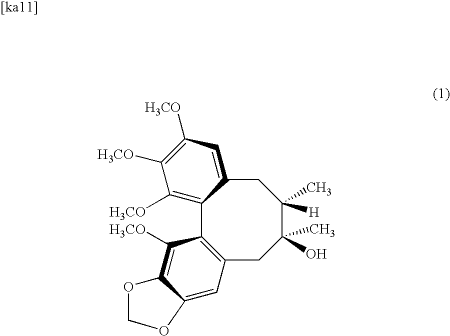

The ATR inhibitor according to the present invention is characterized in that:

ATR inhibitor containing a compound having a following formula (2) as an active constituent.

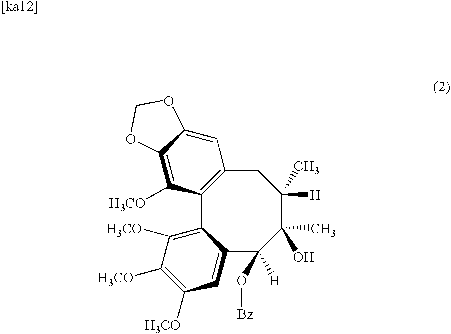

The ATR inhibitor according to the present invention is characterized in that:

ATR inhibitor containing a compound having a following formula (3) as an active constituent.

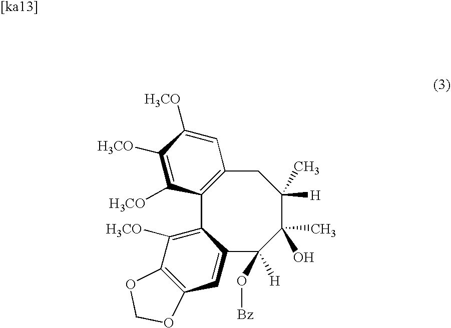

The ATR inhibitor according to the present invention is characterized that:

ATR inhibitor containing a compound having a following formula (4) as an active constituent.

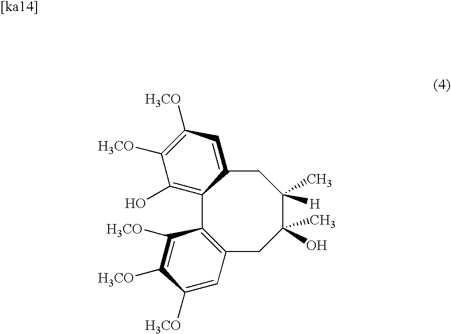

The ATR inhibitor according to the present invention is characterized in that:

ATR inhibitor containing a compound having a following formula (5) as an active constituent.

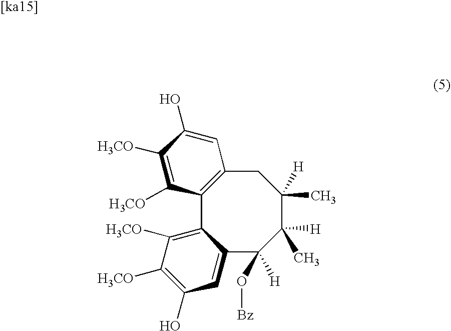

The ATR inhibitor according to the present invention is characterized in that:

ATR inhibitor containing a compound having a following formula (6) as an active constituent.

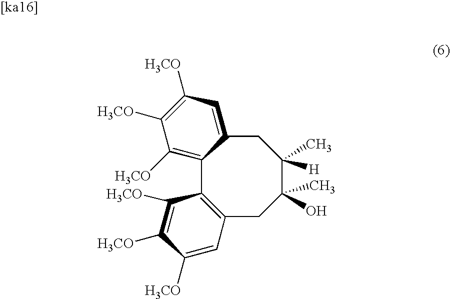

The ATR inhibitor according to the present invention is characterized in that:

ATR inhibitor containing a compound having a following formula (7) as an active constituent.

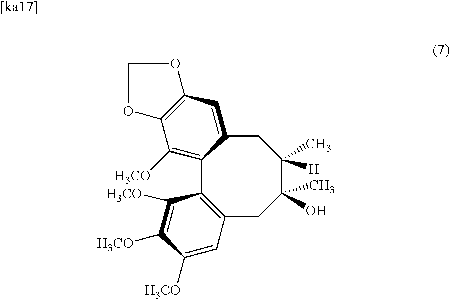

The ATR inhibitor according to the present invention is characterized in that:

ATR inhibitor containing a compound having a following formula (8) as an active constituent.

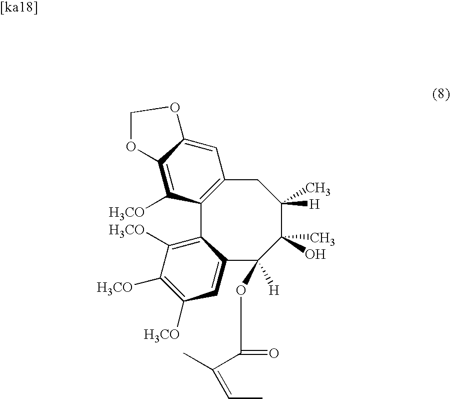

The ATR inhibitor according to the present invention is characterized in that:

ATR inhibitor containing a compound having a following formula (9) as an active constituent.

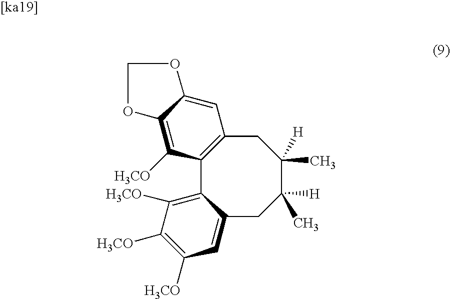

The ATR inhibitor according to the present invention is characterized in that:

ATR inhibitor containing a compound having a following formula (10) as an active constituent.

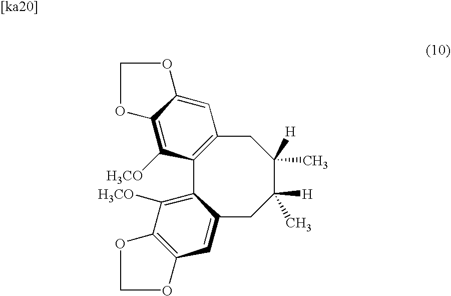

Among these, it is preferable that the ATR inhibitor according to the present invention contains compounds of the Formulas (1) to (5) as an active constituent.

In the ATR inhibitor according to the present invention, it is preferable that the ATR activity is a protein kinase activity.

In the ATR inhibitor according to the present invention, it is preferable that the protein kinase activity is an activity of phosphorylating cell-cycle-related proteins.

In the ATR inhibitor according to the present invention, it is preferable that the cell-cycle-related proteins are p53.

In the ATR inhibitor according to the present invention, it is preferable that the cell-cycle-related proteins are selected from the group consisting of Brca1 and Chk1.

### EFFECT OF INVENTION

According to the present invention, it is possible to specifically inhibit the ATR activity both of in vitro and in vivo.

### BRIEF EXPLANATION OF DRAWING

FIG. 1 is a graph showing the effect of Schizandrin B on the surviving rate of A549 cells irradiated with UV;

FIG. 2 is figures showing the effect of Schizandrin B on the cell cycle of A549 cells irradiated with UV, wherein (A) is a figure showing the content of the phosphorylated Histone H3 in the total cells, and (B) shows ratio of the dividing cells to the total cells based on the content of the phosphorylated histone H3;

FIG. 3 is a graph showing the effect of Schizandrin B on the rate of dividing cells in A549 cells irradiated with ionizing radiation;

FIG. 4 is a graph showing the effect of Schizandrin B on the phosphorylation of p53 in A549 cells irradiated with UV or ionizing radiation;

FIG. 5-1 is a figure showing the dose-depended effect of Schizandrin B on the phosphorylation of p53 in A549 cells irradiated with UV;

FIG. 5-2 is a figure showing the effect of ATR inhibitor according to the present invention on the phosphorylation of p53 in A549 cells irradiated with UV;

FIG. 6 is a figure showing the effect of Schizandrin B on the cell cycle of A549 cells;

FIG. 7 is a graph showing the effect of Schizandrin B on the protein kinase activities of ATR and ATM, wherein (A) corresponds to ATR, and (B) corresponds to ATM;

FIG. 8 is a figure showing the effect of Schizandrin B on the phosphorylation of proteins in AT2KY cells irradiated with UV;

FIG. 9 is a figure showing the effect of Schizandrin B on the phosphorylation of proteins in A549 cells irradiated with UV, wherein the expression of ATR or ATM is inhibited in the cells;

FIG. 10 is a figure showing the effect of Schizandrin B on the phosphorylation of MAPK in A549 cells.

### BEST MODE FOR CARRYING OUT THE INVENTION

The ATR inhibitor according to the present invention can be compounds commercially available including synthetic compounds and natural compounds, and can be manufactured through the well-known synthetic method as disclosed in, e.g. Patent Document 1. In addition, it can be extracted and/or purified from the natural Chinese medicine including Fructus Schisandrae originated from Schisandra Chinensis according to the well-known method as disclosed in, e.g. Patent Document 2. In particular, Schizandrin B (SchB) of formula (1) can be manufactured according to Patent Document 2. That is, Fructus Schisandrae is subjected to extraction with hydrocarbon solvent such as n-hexane or alcoholic solvent such as methanol under heat for a predetermined period. Then, the extract is applied on the column chromatography to obtain a solution containing a mixture of water-insoluble material and water-soluble material, and so obtained solution is concentrated to dryness, followed by adding a mixture of potassium hydroxide and methanol to saponify, thereby obtaining a raw material. Methanol is added to the raw material to solve under stir, followed by dissolving it in aqueous solution of potassium hydroxide. Then, the methanol is removed from obtained solution to precipitate the water-insoluble material of the target compound, thereby obtaining Schizandrin B.

In addition, Gomisin C (GC) can be manufactured according to the method as disclosed in Patent Document 3. That is, after the extraction with a solvent having low polarity such as petroleum ether, it is subjected to the partition extraction using water-insoluble solvent having low polarity such as petroleum ether and n-heptane, and water-soluble solvent having high polarity such as methanol, followed by, if necessary, to treatment such as column chromatography and high-performance liquid chromatography to obtain the target compound.

In addition, Gomisin G (GG) of the formula (3) can be manufactured from the dry stuck of Shizandra arisanensis according to the Non-Patent-Related Document 12 in which it is extracted with ethyl acetate, followed by, if necessary, purifying the target compound with column chromatography.

In addition, Gomisin H (GH) of the formula (4) can be derived from Schizandra chinensis according to Patent Document 5.

In addition, Gomisin J (GJ) of the formula (5) can be manufactured from Schisandrae Fructus according to Patent Document 1, in which deoxyschizandrin extracted from Schisandrae Fructus is acylated to obtain the compound of a formula (B) as mentioned below, then dealkylated (R₁ group of the formula), and deacylated (COR group of the formula) to obtain the target compound.

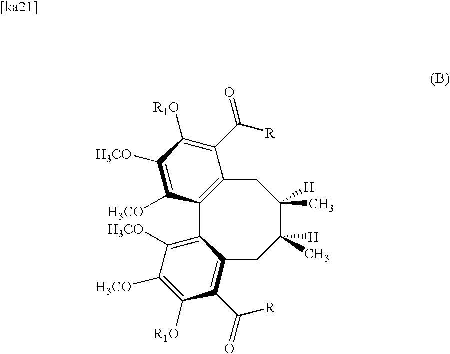

(In the formula, R₁ indicates lower alkyl, and R indicates hydrogen atom or lower alkyl.)

In addition, Schizandrin (Sch) of the formula (6) can be manufactured according to the method as disclosed in Patent Document 3. That is, after the extraction with a solvent having low polarity such as petroleum ether, it is subjected to the partition extraction using water-insoluble solvent having low polarity such as petroleum ether and n-heptane, and water-soluble solvent having high polarity such as methanol, followed by, if necessary, to treatment such as column chromatography and high-performance liquid chromatography to obtain the target compound.

In addition, Gomisin A (GA) can be manufacture according to the method of the Patent Document 3, or can be manufactured according to Patent Document 4, in which five-membered ring of the compound of a formula (A) as mentioned below is reduced to dihydroxylate, followed by subjecting to the oxidation and methanesulphonyl reaction to obtain dl-Schizandrin (III), and then is epimerically extracted to obtain the target compound.

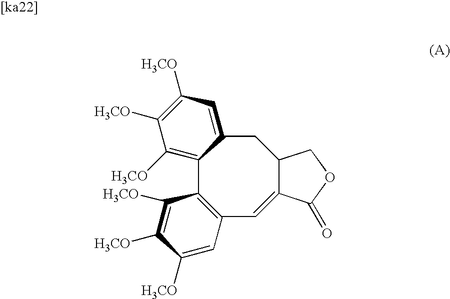

In addition, Gomisin B (GB) of the formula (8) can be manufacture from the dry stuck of Shizandra arisanensis according to the Non-Patent-Related Document 12 in which it is extracted with ethyl acetate, followed by, if necessary, purifying the target compound with column chromatography.

In addition, Gomisin N (GN) of the formula (9) can be derived from Schizandra chinensis according to Patent Document 5.

In addition, Schizandrin C(SC) of the formula (10) can be manufacture from Schisandrae Fructus according to Non-Patent-Related Document 13 in which the extracts of Schisandrae Fructus with petroleum ether are purified with silicagel column chromatography.

### EMBODIMENT

### Embodiment 1

### Effect of Schizandrin B on the Surviving Rate of A549 Cells Irradiated with UV

A549 cells were seeded as 500 cells/60 mm dish in DMEM medium supplemented with 10% fetal bovine serum (FBS), 100 μg/mL of streptomycin and 100 units/mL penicillin (hereinafter, referred to as “growth medium”), and incubated for overnight. Schizandrin B (which is prepared as 10 g/L DMSO solution of Schizandrin B, and is used to dilute it with the medium as a DMSO concentration of less than 5 g/L) was added in the medium as final concentration of 1 μM and 10 μM in the medium.

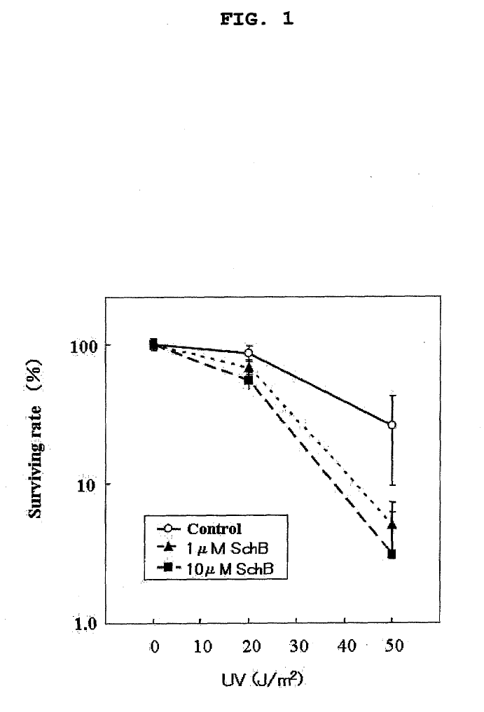

At 1 hour after the addition of Schizandrin B, the medium was removed, and were irradiated with 20 J/m² and 50 J/m² of UV (wavelength 254 nm, the same as follows) using Stratalinker (Stratagene). The medium containing the same concentration of Schizandrin B (hereinafter, referred to as “Schizandrin B-containing medium”) was added. After the consecutive culture for 14 days, the cells were washed with phosphate-buffered-saline (PBS), and stained with 2% methylene blue. The surviving cell numbers were counted and calculated according to the clonogenic assay. The result is shown in FIG. 1. In FIG. 1, the horizontal axis indicates the radiation dose of irradiated UV, and the vertical axis indicates percentage of the surviving ratio of surviving cells of treated group with regard to that of non-treated group. In figures, symbol “◯” indicates the group not treated with Schizandrin B, symbol “▴” indicates the group treated with 1 μM of Schizandrin B, and symbol “▪” indicates the group treated with 10 μM of Schizandrin B. It should be noted that each date are means±S.D. (n=3).

### Embodiment 2-1

### Effect of Schizandrin B on the Cell Cycle of A549 Cells Irradiated with UV

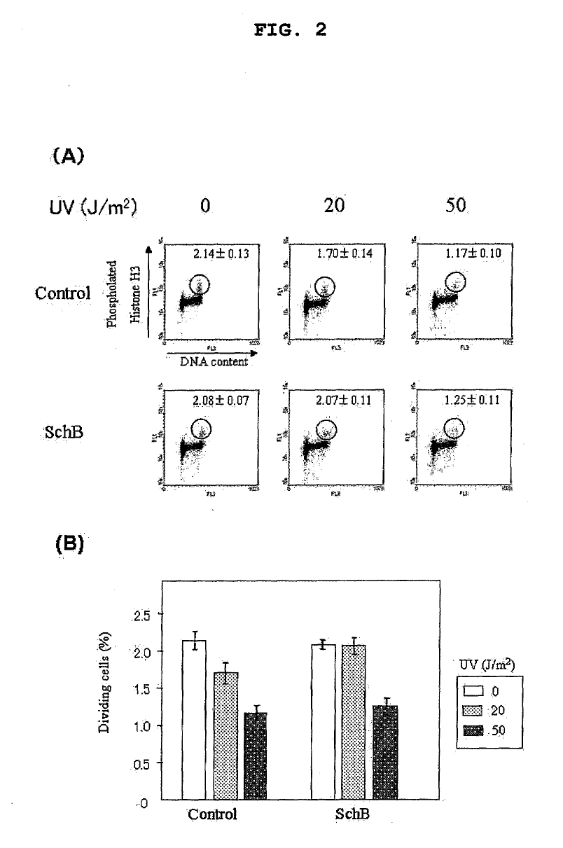

A549 cells were seeded as 3×10⁴ cells/cm² in the growth medium on 10 cm dish and incubated for overnight. Then, Schizandrin B was added to the medium as final concentration of 30 μM. At 1 hour after the addition of Schizandrin B, the medium was removed, and were irradiated with 20 J/m² and 50 J/m² of UV. After the UV irradiation, and then the cells were incubated in the Schizandrin B-containing medium having the same concentration as mentioned above for 1 hour. Then, the percentage of the cells containing phosphorylated histone H3 were measured in accordance with a Measurement of the phosphorylated histone H3 as mentioned below. The result is shown in FIG. 2. The FIG. 2(A) is a result of flowcytometry as obtained in the treatment with Schizandrin B. In the FIG. 2(A), the encircled portion indicates the cell group in G2/M phase, numbers as mentioned above the portion indicates the ratio of the cells in G2/M phase to the total number of cells. In addition, the FIG. 2(B) indicates a graph showing the ratio.

### Embodiment 2-2

Ratios of the cells in G2/M phase to the total number of cells were obtained in accordance with the Embodiment 2-1, except that, in the Embodiment 2-1, 20 J/m² of UV were irradiated instead of 20 J/m² and 50 J/m² of UV and the compounds of the formulas (1) to (10) and Angeloyl Gomisin A of a formula (C) (final concentration of 30 μM) as mentioned below were used instead of the final concentration of 30 μM of Schizandrin B. The result is shown in Table 1.

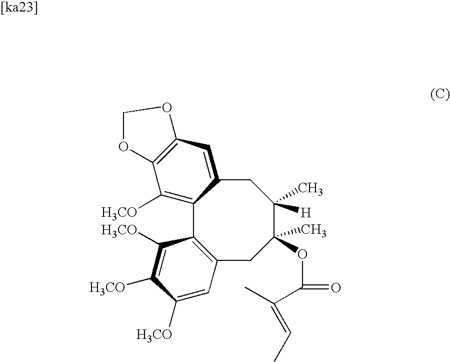

TABLE 1

| | Rate of Divided cells |
|---|---|
| Compounds | (%) |
| Control | 1.88 ± 0.11 |
| UV irradiation | 0.52 ± 0.10 |
| SchC | 0.68 ± 0.11 |
| SchB | 1.09 ± 0.11 |
| S | 0.68 ± 0.04 |
| AGA | 1.04 ± 0.06 |
| GA | 0.90 ± 0.07 |
| GB | 0.85 ± 0.12 |
| GC | 1.16 ± 0.16 |
| GG | 1.13 ± 0.05 |
| GH | 1.42 ± 0.03 |
| GJ | 1.08 ± 0.08 |
| GN | 0.88 ± 0.12 |

### Embodiment 3

### Effect of Schizandrin B on the Rate of Dividing Cells in A549 Cells Irradiated with UV

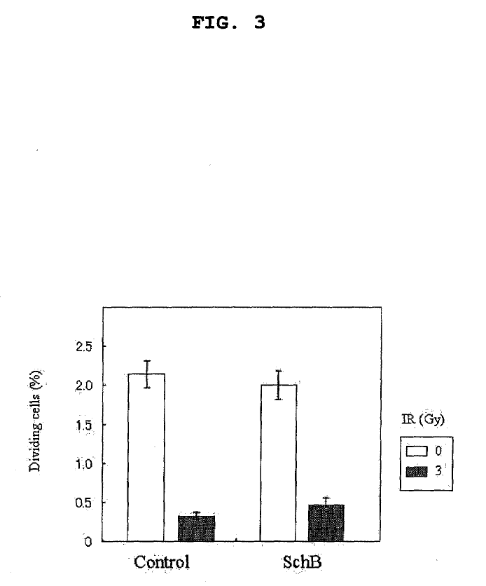

A549 cells were seeded as 3×10⁴ cells/cm² in the growth medium on 10 cm dish, incubated for overnight, and Schizandrin B was added in the medium as final concentration of 30 μM in the medium. After 1 hour incubation, the medium was removed, irradiated with 3 Gy of ionizing radiation, and incubated for 1 hour in the Schizandrin B-containing medium having the same concentration as mentioned above. The Measurement of the cells containing phosphorylated histone H3 as mentioned below was performed for the cells. The result is shown in FIG. 3. Values in the Figure are the same as those of FIG. 2(B).

### Embodiment 4

### Effect of Schizandrin B on the Phosphorylation of p53 in A549 Cells Irradiated with UV or Ionizing Radiation

A549 cells were seeded as 3×10⁴ cells/cm² in the growth medium on 10 cm dish, incubated for overnight, and Schizandrin B was added in the medium as final concentration of 30 μM in the medium. After 1 hour incubation, the medium was removed, irradiated with 20 J/m² of UV, and incubated for 3 hours (hereinafter, referred to as “UV-cell group” with regard to the obtained cells).

In addition, as mentioned above, A549 cells were incubated for overnight, Schizandrin B was added as the final concentration of 30 μM in the medium, and incubated for 1 hour. The medium of the cells was removed, and the cells were irradiated with 10 Gy of ionizing radiation (γ ray), and incubated for 2 hours in the Schizandrin B-containing medium having the same concentration as mentioned above (hereinafter, referred to as “IR-cell group” with regard to the obtained cells).

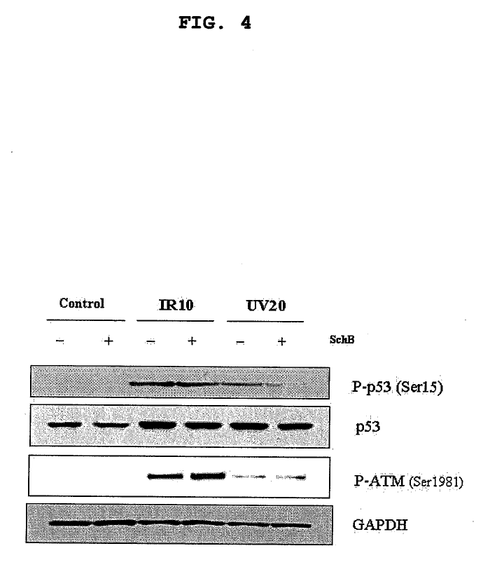

It should be noted that both groups of the UV-cell group and IR-cell group were cultured in the presence of 50 μM of N-acetyl-L-Leucyl-L-Leucyl-L-norleucinal (Sigma, Mo., USA) (hereinafter, referred to as “LLnL”) from 15 minutes prior to the irradiation to collection of the cells as mentioned below. Protein in the cells were extracted in accordance with a Preparation of proteins as mentioned below, immunoblot analysis was performed with regard to the phosphorylated p53 at 15 serine (P-p53 (Ser 15)), p53 protein (p53), phosphorylated ATM protein at 1981 serine (P-ATM (Ser1981)) and glyceraldehyde-3-phosphate dehydrogenase (GAPDH) in accordance with an Immunoblot analysis as mentioned below. The result is shown in FIG. 4.

### Embodiment 5-1

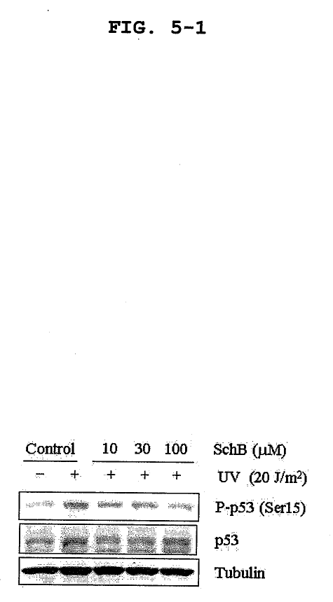

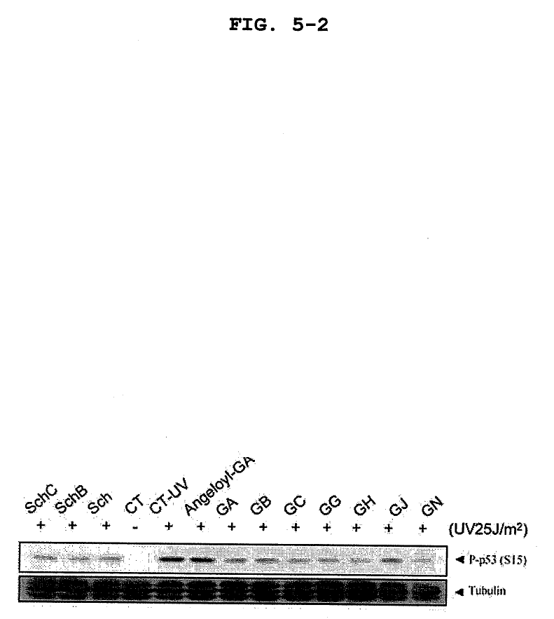

The dose-depended effect of Schizandrin B on the phosphorylation of p53 in A549 cells irradiated with UV A549 cells were seeded as 3×10⁴ cells/cm² in the growth medium on 10 cm dish, incubated for overnight, and Schizandrin B was added in the medium as final concentration of 10, 30 and 100 μM in the medium. After 1 hour incubation, the medium was removed, and the cells were irradiated with 20 J/m² of UV. The cells were treated with LLnL as mentioned in Embodiment 4. Then, expression of p53 and phosphorylated p53 at 15 serine (P-p53 (Ser15)) in the cells treated with the same concentration of Schizandrin B as mentioned above was examined. The result is shown in FIG. 5-1.

### Embodiment 5-2

Expression of phosphorylated p53 at 15 serine (P-p53 (Ser15)) was examined in accordance with Embodiment 5-1, except that, in Embodiment 5-1, compounds of the Formulas (1) to (10) and the compound of the Formula (C) (final concentration of 30 μM) were added in the medium instead of the addition of final concentration of 10, 30 and 100 μM of Schizandrin B in the medium, and 25 J/m² of UV was irradiated instead of the 20 J/m² of UV irradiation. Electrophoresis image as obtained is shown in FIG. 5-2, and relative ratio of the band intensity corresponding to P-p53 (Ser15) to that of the band as obtained in the irradiation with UV as 100% is shown in Table 2.

TABLE 2

| | Relative rate |
|---|---|
| Compounds | (%) |
| UV irradiation | 100.0 |
| Control | 15.4 |
| SchC | 42.6 |
| SchB | 37.9 |
| S | 42.7 |
| AGA | 92.2 |
| GA | 58.3 |
| GB | 60.9 |
| GC | 49.8 |
| GG | 51.3 |
| GH | 50.3 |
| GJ | 61.1 |
| GN | 38.4 |

### Embodiment 6

### Effect of Schizandrin B on the Cell Cycle of A549 Cells

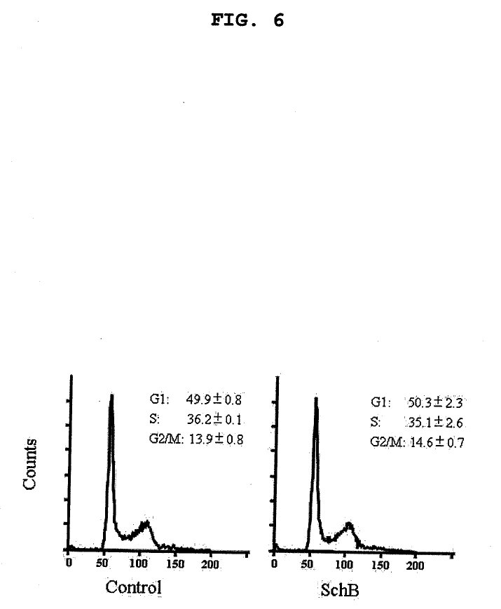

A549 cells were seeded as 3×10⁴ cells/cm² in the growth medium on 10 cm dish, incubated for overnight, and Schizandrin B was added in the medium as final concentration of 30 μM in the medium. After 4 hours of the addition, the cell cycle of the cells was examined in accordance with a FACS analysis as mentioned below. The result of the FACS analysis is shown in FIG. 6.

### Embodiment 7-1

### Effect of Schizandrin B on the Protein Kinase Activity of ATR and ATM

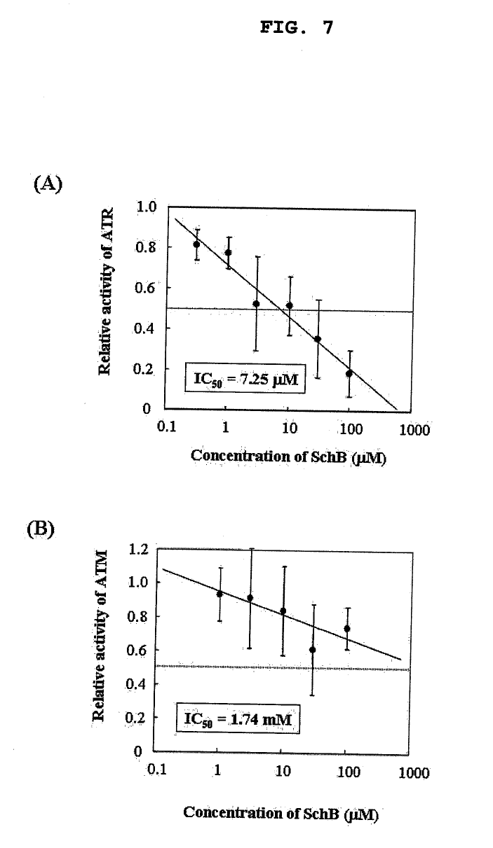

1 μg of recombinant PHAS-I protein (Alexis Biochemicals) as a substrate, and Schizandrin B were added to mixtures of ATR and ATM for the kinase reaction as prepared in accordance with a Preparation of mixtures of ATR and ATM for kinase reaction as follows. 300 μM of ATP containing a suitable concentration of ³²P-ATP was added to react it for 20 minutes at 30° C. 4 volumes of SDS sample loading buffer (200 mM Tris-HCl (pH 6.8), 400 mM DTT and 8% SDS) were added to the reaction mixture to stop the reaction, and the obtained mixture is applied to 12% SDS-PAGE. After the gel as obtained was dried, γ ray corresponding to the band of ³²P—PHAS-I in the mixture was evaluated using autoradiography method. The result is shown in FIG. 7. In the Figure, (A) and (B) indicate the relative activities of ATR and ATM, respectively, the horizontal axis indicates the concentration of Schizandrin B, and the vertical axis indicates the relative value of the count as obtained in Schizandrin B-treated group to that of no-addition of Schizandrin B group.

### Embodiment 7-2

Effect of the ATR Inhibitor According to the Present Invention on the Kinase Activity of ATR

IC₅₀s of the compounds for the phosphorylating activity of ATR were estimated based on the lines as obtained in accordance with Embodiment 7-1, except that, in Embodiment 7-1, the compounds of the Formulas (1) to (5) and the compound of the Formula (C) were used instead of Schizandrin B. The values are shown in Table 3.

TABLE 3

| Compounds | IC₅₀ (μM) |
|---|---|
| SchB | 7.25 |
| AGA | 110.78 |
| GC | 10.63 |
| GG | 32.00 |
| GH | 45.68 |
| GJ | 67.81 |

### Embodiment 8

### Effect of Schizandrin B on the Phosphorylation of Proteins in AT2KY Cells Irradiated with UV

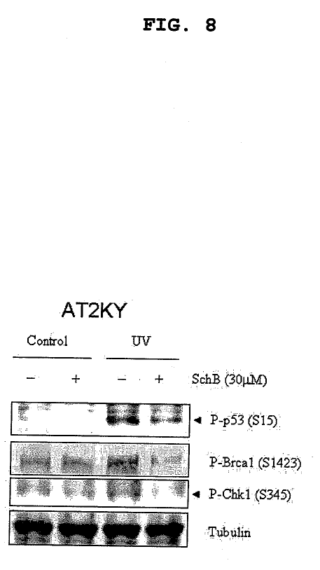

AT2KY cells (Health Science Research Resources Bank, Osaka, Japan) were seeded as 3×10⁴ cells/cm² in RPMI1640 supplemented with 15% FBS, 100 μg of streptomycin and 100 units/mL of penicillin on 10 cm dish, and incubated for overnight. Schizandrin B was added in the medium as final concentration of 30 μM in the medium. After 1 hour of the addition, the medium was removed, and 20 J/m² of UV was irradiated. Then, expression of phosphorylated p53 at 15 serine (P-p53 (Ser15)), phosphorylated Brca1 at 1423 Serine (P-Brca1 (S1423)) and phosphorylated Chk1 at 345 serine in the cells treated with the same concentration of Schizandrin B as mentioned above for 4 hours were examined in accordance with the immunoblot analysis as mentioned below. The result is shown in FIG. 8.

### Embodiment 9

### Effect of Schizandrin B on the Phosphorylation of Proteins in A549 Cells Irradiated with UV, wherein the Expression of ATR or ATM is Inhibited in the Cells

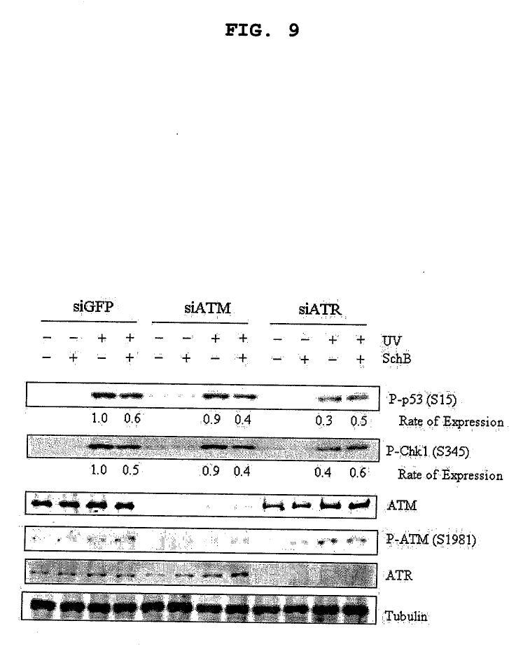

A549 cells were seed as 3×10⁴ cells/cm² in the growth medium on 10 cm dish, and incubated for overnight. The transfection of siRNAs (siGFP, siATM and siATR) as obtained in accordance with a Preparation of siRNA as mentioned below were performed in the cells using Oligofectamine (Invitrogen) and Opti-MEM (Invitrogen) with manufacture's guidance. In brief, after the incubation for overnight, the medium was replaced with a fresh growth medium for another 72 hours, the cells were treated with 30 μM of Schizandrin B for 1 hour, the medium was removed, and the cells were irradiated with 20 J/m² of UV. The phosphorylation of p53 at 15 serine (P-p53 (Ser15)), Chk1 at 345 Serine (P-Chk1 (S345)), and ATM protein at 1981 Serine (P-ATM(Ser1981)), and expression of ATM protein (ATM) and ATR protein (ATR) in the cells treated with the same concentration of Schizandrin B as mentioned above for 3 hours were examined by immunoblot analysis as mentioned below. The result is shown in FIG. 9. It should be noted that, in FIG. 9, the expression ratios as noted below the columns of P-p53(S15) and P-Chk1(S345) indicate the relative band intensities to those as obtained in siGFP and UV of control group as 1.0.

### Embodiment 10

### Effect of Schizandrin B on the Phosphorylation of MAPK in A549 Cells

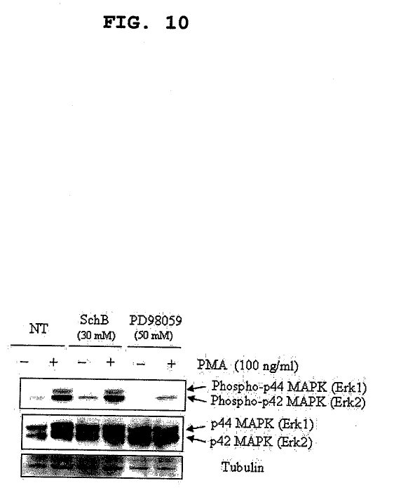

A549 cells were seeded as 3×10⁴ cells/cm² in the growth medium on 10 cm dish, incubated for overnight, and further incubated for 16 hours in the serum-free growth medium. Then, the cells were treated with 50 μM of PD098059 (Sigma) or 30 μM of Schizandrin B, and further treated with 100 ng/mL of phorbol 12-myristate 13-acetate (PMA; Sigma) for 5 minutes. The expressions of 42 kDa and 44 kD of mitogen activated protein kinase (p42 MAPK and p44 MAPK) and their phosphorylation were evaluated. The result is shown in FIG. 10.

(Measurement of the Phosphorylated Histone H3)

The cells were harvested and fixed with 70% ice-cooled ethanol after the washing with PBS. PBS containing 0.25% Triton X-100 was added, and the cells were stood on ice for 30 minutes. Cells were centrifuged (500×g, 5 minutes) and replaced to PBS containing 1% bovine serum albumin (BSA) and 1 μg of anti-rabbit serum (specifically binding to the phosphorylated histone H3 at 13 Serine), and incubated for 4 hours at room temperature.

After the subsequent washes with PBS containing 1% BSA, goat anti-rabbit IgG antibody conjugated with FITC which diluted with 100 times with PBS containing 1% BSA was added, and incubated for 30 minutes at dark place. These cells were counterstained with PI in accordance with the FACS analysis as mentioned below. The percentage of cells containing phosphorylated histone H3 was measured and evaluated with flowcytometry (Beckman-Coulter).

(Immunoblot Analysis)

50 μg of cellular proteins (as prepared in accordance with the Preparation of proteins as mentioned below) were applied to SDS polyacrylamide gel electrophoresis (12.5%), the electrophored proteins were transferred into the nitrocellulose membrane in accordance with the standard method, and blocking reaction was performed for 1 hour at room temperature using Tris-buffered saline (TBS-T) containing 5% skim milk and 0.1% Triton X-100. So obtained membrane were incubated with the desired adequate antibodies as mentioned below for 16 hours at 4° C., and then incubated with secondary anti-serum (corresponding to the animal originated from the desired antibodies) conjugated with horseradish peroxidase for 1 hour at room temperature. Then, the target proteins were visualized with ECL reaction kit (Amersham) and chemiluminescence film (Amersham) to obtain a protein image.

<Antibodies>

p53 antibody (Calbiochem)

Phosphorylated p53 antibody

(Cell Signaling Technology)

Phosphorylated ATM antibody (Rockland)

Phosphorylated Chk1 antibody

(Cell Signaling Technology)

ATR antibody (Bethyl)

Tubulin antibody (Sigma)

GAPDH antibody (Santa Cruz)

Phosphorylated Brca1 antibody (Upstate)

Anti-MAPK antibody and anti-phosphorylated MAPK antibody (Cell Signaling)

(Preparation of Proteins)

The cells were harvested with ice-cooled PBS from the culture dish, and were washed twice with cooled PBS. Then, it was centrifuged to obtain cell pellet. The pellet was solubilized with UTB buffer as mentioned below to prepare cellular protein. The content of the proteins was measured with Protein Assay kit (Bio-Rad).

### <UTB Buffer>

| | |
|---|---|
| 8 mM | Urea |
| 150 mM | 2-mercaptoethanol |
| 50 mM | Tris (pH 7.5) |

### (Facs Analysis)

After washing the cells with PBS, the cells were fixed with 70% ice-cooled ethanol. The pellet as colleted with centrifugation was washed with PBS, and further centrifuged. Cells were re-suspended with a solution containing 100 μM final concentration of propidium iodide (Sigma, hereinafter, referred to as “PI”) and 40 μg/mL of RNase A (PI solution), and incubated for 30 minutes at room temperature. Then, the cell cycle analysis was performed with flowcytometry (Beckman-Coulter).

(Preparation of Mixtures of ATR and ATM for Kinase Reaction)

ATR conjugated with Flag as constructed with conventional method as disclosed in, e.g. Non-Patent-Related Document 9 was used for a mixture of ATR for kinase reaction. In detail, Bam H1H-Swa I fragment of ATR gene (Sequence Number 4) (1 kb) was substituted with a gene having the ATR gene and Flag (KDDDDK) at N terminal and the gene was amplified with PCR. So obtained gene product was introduced into pcDNA3.1, and target product was obtained from the plasmid. In addition, ATM conjugated with Flag as manufactured in accordance with the well-known method as disclosed in e.g. non-patent related documents 10 and 11 and using Quikchange site-Directed Mutagenesis Kit (Stratagene) was used for a mixture of ATM for kinase reaction.

These ATR and ATM plasmids tagged with Flag (Sequence Numbers 4 and 5) were transfected in 293T cells (ATCC: CRL-11268) by Calcium phosphate method as followed. After 2 days, 5 mg of cellular proteins were prepared using IP buffer in accordance with the Preparation of proteins as mentioned below instead of the UTB buffer, and ATR and ATM were immuno-precipitated with 20 μg of anti-Flag M2 monoclonal antibody (Sigma) for 4 hours at 40, independently. The precipitates were incubated with beads conjugated with protein G cepharose (Amersham). The obtained immuno-complexes were washed twice with a TGN buffer, and further washed once with a Kinase buffer as mentioned below to obtain Mixtures of ATR and ATM for kinase reaction (Sequence Numbers 6 and 7).

<IP Buffer>

| | |
|---|---|
| 10 mM | Tris (pH 7.5) |
| 1 mM | EDTA |
| 1 mM | EGTA |
| 150 mM | NaCl |
| 0.5% | NP-40 |
| 1% | Triton X-100 |
| 1 mM | Phenylmethane sulfonylfluoride (PMSF) |
| 2 μg/mL | Pepstatin |
| 2 μg/mL | Aprotinin |
| 1 mM | p,p′- |
| | dichlorodiphenyltrichloroethane(DDT) |

<TGN Buffer>

| 50 mM | Tris (pH 7.4) |
|---|---|
| 50 mM | Glycerophosphoric acid |
| 150 mM | NaCl |
| 1% | Tween20 |
| 10% | Glycerol |

<Kinase Buffer>

| | |
|---|---|
| 10 mM | Hepes (pH 7.5) |
| 50 mM | Glycerophosphoric acid |
| 50 mM | NaCl |
| 10 mM | MgCl₂ |
| 10 mM | MnCl₂ |

<Calcium Phosphate Method>

293T cells were seed as 2×10⁴ cells/cm² in the growth medium on 10 cm dish, and incubated for overnight. The above-mentioned Flag-ATM or Flag-ATR mixed with 25 mM final concentration of BES (N,N-bis(2-hydroxyethyl)-2-aminoethanesulfonic acid) and 25 mM of CaCl₂ were added to the cell cultures. After the incubation for overnight, the medium was replaced with a fresh growth medium and incubated for 2 days.

(Preparation of siRNA)

The Double strand siRNAs for ATR, ATM and green fluorescence protein (GFP) (corresponding to Sequence Numbers 1, 2 and 3, respectively) were purchased from Qiagen (HP GenomeWide siRNA). The double strand siRNAs were referred to as siATR, siATM and siGFP.

(Discussion)

ATM (ataxia telangiectsia mutated) and ATR (ataxia telangiectasia and Rad-3-related) are playing major role in cellular DNA damage responsive signal transduction of cell cycle. ATM and ATR phosphorylate the so-called checkpoint kinases and transcriptional factors such as Chk1, p53, NBS1, Brca1 and SMC1. In order to avoid carrying over the DNA damage to the next generation causing to carcinogenesis, these checkpoint signals are regulating the overall of cell cycle. The deficiency of ATM or ATR decreases the cell viability after DNA damage induced by UV, IR irradiation or damage inducible agents. This shows the requirements of ATM and ATR for the repair, maintenance and management of damaged DNA. In the present invention, the present applicant has demonstrated that Schizandrin B, an ingredient of Schisandra Chinensis, dose-dependently decreased the cell viability after UV irradiation. It indicates that Schizandrin B has an inhibitory effect on checkpoint signal transduction pathways in the process of DNA damage response in vivo.

Although Histone H3 is phosphorylated at 10 serine in unperturbed mitotic cells and it is an index for entering from G2 phase into M phase, DNA damages lead to decrease of the ratio of phosphorylated Histone H3 positive cells in the case that the G2/M checkpoint is effective. According to FIG. 2, the ratio of the phosphorylated Histone H3 has decreased in the cells irradiated with 20 and 50 J/m² of UV. On the other hand, Schizandrin B of the formula (1) remarkably disrupted the G2/M checkpoint at 20 J/m² of UV irradiation, though it was not significant at 50 J/m² of UV. Indeed, in the low dose of UV irradiation, G2/M checkpoint was completely abolished by Schizandrin B during DNA damage response. In addition, G2/M checkpoint was completely abolished by the compounds of the formulas (1) to 10 and the compound of the formula (C). On the other hand, the dramatic inhibition of G2/M checkpoint did not occur in the cells irradiated with IR. It has been known that IR irradiation activates ATM and UV irradiation activates ATR, thereby going on the cell cycle checkpoints. These results suggest that Schizandrin B has an inhibitory effect on ATR activity rather than ATM in G2/M checkpoint during DNA damage induced low dose of UV irradiation. The same can apply to the ATR inhibitor according to the present invention such as the compounds of the formulas (1) to (10).

To clarify the inhibitory effect of the ATR inhibitor according to the present invention on ATR kinase activity in vivo, the p53 phosphorylation after DNA damage induced by IR or UV irradiation was examined. It is widely recognized that p53 is phosphorylated at multiple sites in vivo by several different kinds of protein kinases and that p53 is known as very important transcriptional factor in the cells. Considering the function of p53 in DNA damage response during cancer therapy, p53 is specifically phosphorylated at serine 15 by ATR and ATM when the cells are damaged by UV irradiation, γIR irradiation and methylating agent. In the present invention, p53 phosphorylation was decreased by Schizandrin B after 20 J/m² UV irradiation toward A549 cells, while it was not decreased after IR irradiation. The clear activation of ATM at serine 1981 was observed after IR irradiation, and slight activation after 20 J/m² of irradiation was observed. However, Schizandrin B did not influence on the ATM activity. In addition, any localized accumulation and bias of the cell cycle were not observed by Schizandrin B. Further, the inhibition of p53 phosphorylation was observed by the compounds of the formulas (1) to (10). These results indicate that the phosphorylation of p53 at serine 15 by 20 J/m² of UV irradiation is reduced only by ATR.

Non-Patent-Related Document 7 discloses an inhibitory effect of caffeine on ATM and ATR protein kinase activity of A549 adeno carcinoma cells. The present applicant has also examined the effect of Schizandrin B on ATM and ATR protein kinase activities in vitro with identical method of the document. Schizandrin B obviously inhibited the kinase activity of ATR rather than that of ATM, although the incubation of Schizandrin B with the kinase reaction buffer inhibited both ATM and ATR. Indeed, the IC₅₀ of ATR by Schizandrin B was about 240 fold against that of ATM. It has remarkably observed the inhibition of ATR protein kinase activity by the compounds of the formulas (1) to (5). These results of the cell viability and the kinase activity indicate that the ATR protein kinase activity was inhibited during the response of UV irradiation induced DNA damage of the cells.

To examine the effect of Schizandrin B on DNA damage checkpoints, fibroblasts derived from AT patient (AT2KY cells) and cells treated with siRNA were used. UV irradiation of AT2KY cells increased not only phosphorylated p53 but also phosphorylated Brca1 (Ser 1423) and Chk1 (Ser 345) as downstream molecules of ATM and ATR on the checkpoint pathway. Moreover, decreased phosphorylation of p53 and Chk1 by Schizandrin B was only seen in the cells treated with siRNA for control and ATM. In addition, the cells treated with siRNA did not show reduced phosphorylation of checkpoint proteins. These results indicate that Schizandrin B inhibited signal transduction pathways of DNA damage checkpoints in vivo through the inhibition of ATR protein kinase activity.

To verify the specificity of Schizandrin B on ATR inhibition in vivo, it was examined the effect of Schizandrin B on the phosphorylation of ERK, a mitogen activated protein kinase (MAPK) by its specific activation system with serum stimulation and PMA treatment. The phosphorylation of ERK was strongly enhanced by the referenced method. The treatment of Schizandrin B did not affect on ERK phosphorylation. Therefore, the efficacy of Schizandrin B in DNA damage response is highly specific to ATR activity.

In the present invention, the effects of Schizandrin B on DNA damage response induced by UV irradiation to the cells were demonstrated. Since the knocking down of ATR leads to lethal in embryo, it is speculated that ATR is playing crucial role in the developmental stages of mammals. This indicates that ATR is necessary for the checkpoint to the instability of chromatin and the deficiency of the checkpoint caused by the radical continuously created in the body, and further to replicative stress of the cellular DNA during development. On the other hand, ATR maintains the checkpoint in the cells of AT patient (cells genetically inactive of ATM), since ATR also works as a backup molecule of ATM. These indicate that ATR protein kinase is very important in the proliferating cells including tumor incidence. The finding as obtained in the present invention enables to make advantages of anti-cancer therapy such as radiation and chemotherapy since Schizandrin B is capable of abolishing the DNA damaged check points in patients through the inhibition of ATR kinase activity. Caffeine preferentially has ⅕ IC₅₀ for ATM than that of ATR. Therefore; it is speculated that single treatment of Schizandrin B, or the combined treatment of Schizandrin B with caffeine in the radiotherapy and anticancer drug therapy could increase the radio sensitivity in the human clinical cancer therapy.

Non-Patent-Related Document 3 reported the application of caffeine to clinical trial as an assistant of chemotherapy and radiochemotherapy. The document indicates that caffeine-assisted therapy reduced or minimized the size of osteosarcoma than individual treatment of chemotherapy or radiochemotherapy. These results indicate that caffeine inhibited ATM and ATR protein kinase activities, and resulting in the inhibition of the DNA damage checkpoint whichever in proliferating tumor cell cycle. These raise a possibility that Schizandrin B is applicable on clinical trial as Schizandrin B assistant chemotherapy or radiochemotherapy. For further investigation, it is required to estimate whether the elimination or minimization of tumor size can be streamlined by the combined effect of Schizandrin B, along with the clinical test of caffeine. Notably, Schizandrin B is an ingredient of natural plant extraction with expected no or less side effects in the patients. The combination of Schizandrin B with caffeine for chemo/radio therapy would be expected to reduce the amount of administration of chemo/radio therapy for patient with decreased side effects since these assistants are expected to increase the efficiency of chemo/radio therapy with lower dosage.

The present invention will be explained with reference to the preferable embodiments. It is obvious that modifications and alterations to these embodiments can be made without departing from the spirit and scope of the invention as defined in the claims, although it has been explained with the specific embodiments. That is, it should not be construed to limit the present invention to the details of the specific embodiments and attached drawing.

1. ATR inhibitor containing a compound having a following formula (1) as an active constituent.

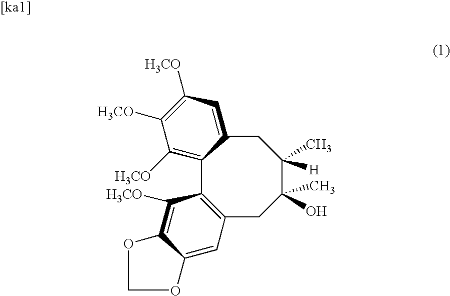

2. ATR inhibitor containing a compound having a following formula (2) as an active constituent.

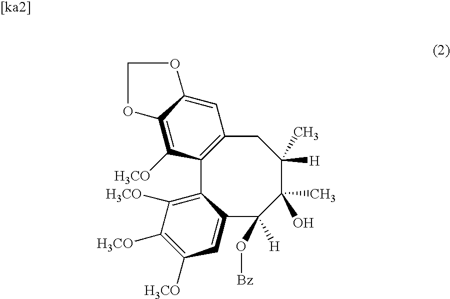

3. ATR inhibitor containing a compound having a following formula (3) as an active constituent.

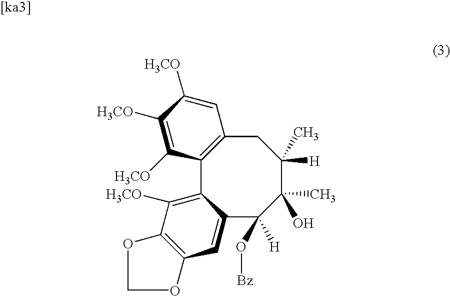

4. ATR inhibitor containing a compound having a following formula (4) as an active constituent.

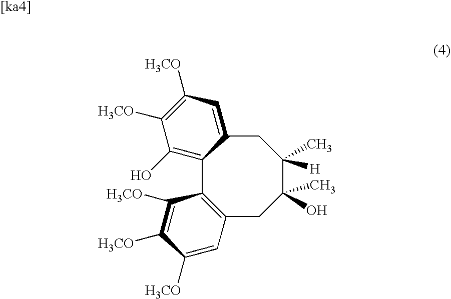

5. ATR inhibitor containing a compound having a following formula (5) as an active constituent.

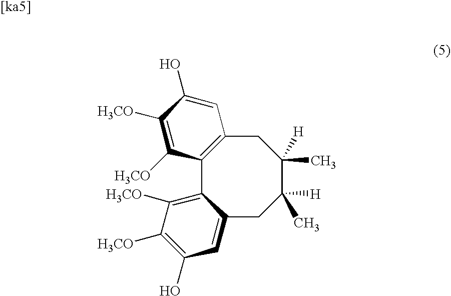

6. ATR inhibitor containing a compound having a following formula (6) as an active constituent.

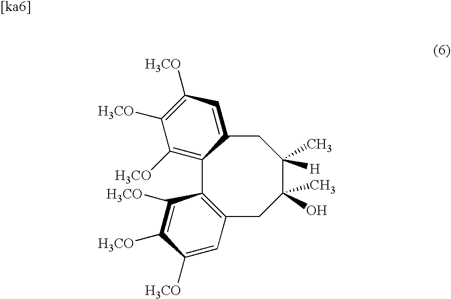

7. ATR inhibitor containing a compound having a following formula (7) as an active constituent.

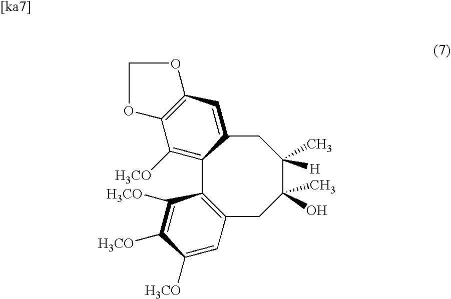

8. ATR inhibitor containing a compound having a following formula (8) as an active constituent.

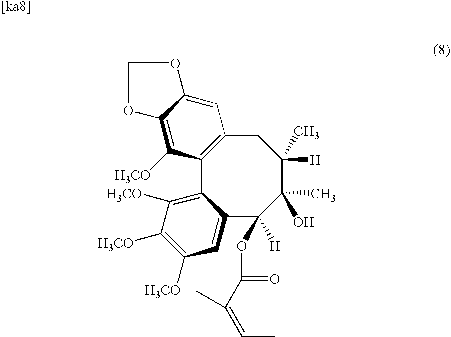

9. ATR inhibitor containing a compound having a following formula (9) as an active constituent.

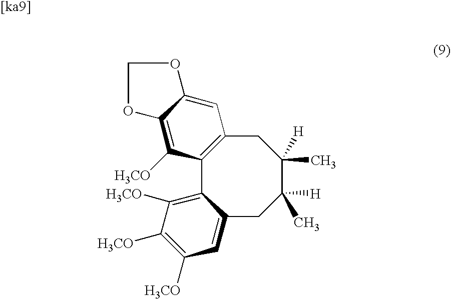

10. ATR inhibitor containing a compound having a following formula (10) as an active constituent.

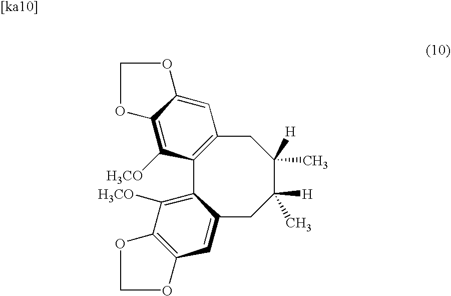

11. The ATR inhibitor as claimed in claim 1, wherein the ATR activity is a protein kinase activity.

12. The ATR inhibitor as claimed in claim 11, wherein said protein kinase activity is an activity of phosphorylating cell-cycle-related proteins.

13. The ATR inhibitor as claimed in claim 12, wherein said cell-cycle-related protein is p53.

14. The ATR inhibitor as claimed in claim 12, wherein said cell-cycle-related proteins are selected from the group consisting of Brca1 and Chk1.


SEQUENCE LISTING (7 sequences)

SEQ ID NO: 1 - DNA, 21 bp, Artificial Sequence
  Description of Artificial Sequence Synthetic oligonucleotide
aagcgcctgattcgagatcct

SEQ ID NO: 2 - DNA, 21 bp, Artificial Sequence
  Description of Artificial Sequence Synthetic oligonucleotide
aagacggtgtgctcatgcggc

SEQ ID NO: 3 - DNA, 24 bp, Artificial Sequence
  Description of Artificial Sequence Synthetic oligonucleotide
cggcaagctgaccctgaagttcat

SEQ ID NO: 4 - DNA, 8265 bp, Artificial Sequence
  Description of Artificial Sequence Synthetic polynucleotide
gcctccacacggctccgtcgggcgccgcgctcttccggcagcggtacgtttggagacgccgggaacccgcgttggcgtggttgactagtgcctcgcagcctcagcatgggggaacatggcctggagctggcttccatgatccccgccctgcgggagctgggcagtgccacaccagaggaatataatacagttgtacagaagccaagacaaattctgtgtcaattcattgaccggatacttacagatgtaaatgttgttgctgtagaacttgtaaagaaaactgactctcagccaacctccgtgatgttgcttgatttcatccagcatatcatgaaatcctccccacttatgtttgtaaatgtgagtggaagccatgagcgcaaaggcagttgtattgaattcagtaattggatcataacgagacttctgcggattgcagcaactccctcctgtcatttgttacacaagaaaatctgtgaagtcatctgttcattattatttctttttaaaagcaagagtcctgctatttttggggtactcacaaaagaattattacaactttttgaagacttggtttacctccatagaagaaatgtgatgggtcatgctgtggaatggccagtggtcatgagccgatttttaagtcaattagatgaacacatgggatatttacaatcagctcctttgcagttgatgagtatgcaaaatttagaatttattgaagtcactttattaatggttcttactcgtattattgcaattgtgttttttagaaggcaagaactcttactttggcagataggttgtgttctgctagagtatggtagtccaaaaattaaatccctagcaattagctttttaacagaactttttcagcttggaggactaccagcacaaccagctagcacttttttcagctcatttttggaattattaaaacaccttgtagaaatggatactgaccaattgaaactctatgaagagccattatcaaagctgataaagacactatttccctttgaagcagaagcttatagaaatattgaacctgtctatttaaatatgctgctggaaaaactctgtgtcatgtttgaagacggtgtgctcatgcggcttaagtctgatttgctaaaagcagctttgtgccatttactgcagtatttccttaaatttgtgccagctgggtatgaatctgctttacaagtcaggaaggtctatgtgagaaatatttgtaaagctcttttggatgtgcttggaattgaggtagatgcagagtacttgttgggcccactttatgcagctttgaaaatggaaagtatggaaatcattgaggagattcaatgccaaactcaacaggaaaacctcagcagtaatagtgatggaatatcacccaaaaggcgtcgtctcagctcgtctctaaacccttctaaaagagcaccaaaacagactgaggaaattaaacatgtggacatgaaccaaaagagcatattatggagtgcactgaaacagaaagctgaatcccttcagatttcccttgaatacagtggcctaaagaatcctgttattgagatgttagaaggaattgctgttgtcttacaactgactgctctgtgtactgttcattgttctcatcaaaacatgaactgccgtactttcaaggactgtcaacataaatccaagaagaaaccttctgtagtgataacttggatgtcattggatttttacacaaaagtgcttaagagctgtagaagtttgttagaatctgttcagaaactggacctggaggcaaccattgataaggtggtgaaaatttatgatgctttgatttatatgcaagtaaacagttcatttgaagatcatatcctggaagatttatgtggtatgctctcacttccatggatttattcccattctgatgatggctgtttaaagttgaccacatttgccgctaatcttctaacattaagctgtaggatttcagatagctattcaccacaggcacaatcacgatgtgtgtttcttctgactctgtttccaagaagaatattccttgagtggagaacagcagtttacaactgggccctgcagagctcccatgaagtaatccgggctagttgtgttagtggattttttatcttattgcagcagcagaattcttgtaacagagttcccaagattcttatagataaagtcaaagatgattctgacattgtcaagaaagaatttgcttctatacttggtcaacttgtctgtactcttcacggcatgttttatctgacaagttctttaacagaacctttctctgaacacggacatgtggacctcttctgtaggaacttgaaagccacttctcaacatgaatgttcatcttctcaactaaaagcttctgtctgcaagccattccttttcctactgaaaaaaaaaatacctagtccagtaaaacttgctttcatagataatctacatcatctttgtaagcatcttgattttagagaagatgaaacagatgtaaaagcagttcttggaactttattaaatttaatggaagatccagacaaagatgttagagtggcttttagtggaaatatcaagcacatattggaatccttggactctgaagatggatttataaaggagctttttgtcttaagaatgaaggaagcatatacacatgcccaaatatcaagaaataatgagctgaaggataccttgattcttacaacaggggatattggaagggccgcaaaaggagatttggtaccatttgcactcttacacttattgcattgtttgttatccaagtcagcatctgtctctggagcagcatacacagaaattagagctctggttgcagctaaaagtgttaaactgcaaagttttttcagccagtataagaaacccatctgtcagtttttggtagaatcccttcactctagtcagatgacagcacttccgaatactccatgccagaatgctgacgtgcgaaaacaagatgtggctcaccagagagaaatggctttaaatacgttgtctgaaattgccaacgttttcgactttcctgatcttaatcgttttcttactaggacattacaagttctactacctgatcttgctgccaaagcaagccctgcagcttctgctctcattcgaactttaggaaaacaattaaatgtcaatcgtagagagattttaataaacaacttcaaatatattttttctcatttggtctgttcttgttccaaagatgaattagaacgtgcccttcattatctgaagaatgaaacagaaattgaactggggagcctgttgagacaagatttccaaggattgcataatgaattattgctgcgtattggagaacactatcaacaggtttttaatggtttgtcaatacttgcctcatttgcatccagtgatgatccatatcagggcccgagagatatcatatcacctgaactgatggctgattatttacaacccaaattgttgggcattttggctttttttaacatgcagttactgagctctagtgttggcattgaagataagaaaatggccttgaacagtttgatgtctttgatgaagttaatgggacccaaacatgtcagttctgtgagggtgaagatgatgaccacactgagaactggccttcgattcaaggatgattttcctgaattgtgttgcagagcttgggactgctttgttcgctgcctggatcatgcttgtctgggctcccttctcagtcatgtaatagtagctttgttacctcttatacacatccagcctaaagaaactgcagctatcttccactacctcataattgaaaacagggatgctgtgcaagattttcttcatgaaatatattttttacctgatcatccagaattaaaaaagataaaagccgttctccaggaatacagaaaggagacctctgagagcactgatcttcagacaactcttcagctctctatgaaggccattcaacatgaaaatgtcgatgttcgtattcatgctcttacaagcttgaaggaaaccttgtataaaaatcaggaaaaactgataaagtatgcaacagacagtgaaacagtagaacctattatctcacagttggtgacagtgcttttgaaaggttgccaagatgcaaactctcaagctcggttgctctgtggggaatgtttaggggaattgggggcgatagatccaggtcgattagatttctcaacaactgaaactcaaggaaaagattttacatttgtgactggagtagaagattcaagctttgcctatggattattgatggagctaacaagagcttaccttgcgtatgctgataatagccgagctcaagattcagctgcctatgccattcaggagttgctttctatttatgactgtagagagatggagaccaacggcccaggtcaccaattgtggaggagatttcctgagcatgttcgggaaatactagaacctcatctaaataccagatacaagagttctcagaagtcaaccgattggtctggagtaaagaagccaatttacttaagtaaattgggtagtaactttgcagaatggtcagcatcttgggcaggttatcttattacaaaggttcgacatgatcttgccagtaaaattttcacctgctgtagcattatgatgaagcatgatttcaaagtgaccatctatcttcttccacatattctggtgtatgtcttactgggttgtaatcaagaagatcagcaggaggtttatgcagaaattatggcagttctaaagcatgacgatcagcataccataaatacccaagacattgcatctgatctgtgtcaactcagtacacagactgtgttctccatgcttgaccatctcacacagtgggcaaggcacaaatttcaggcactgaaagctgagaaatgtccacacagcaaatcaaacagaaataaggtagactcaatggtatctactgtggattatgaagactatcagagtgtaacccgttttctagacctcataccccaggatactctggcagtagcttcctttcgctccaaagcatacacacgagctgtaatgcactttgaatcatttattacagaaaagaagcaaaatattcaggaacatcttggatttttacagaaattgtatgctgctatgcatgaacctgatggagtggccggagtcagtgcaattagaaaggcagaaccatctctaaaagaacagatccttgaacatgaaagccttggcttgctgagggatgccactgcttgttatgacagggctattcagctagaaccagaccagatcattcattatcatggtgtagtaaagtccatgttaggtcttggtcagctgtctactgttatcactcaggtgaatggagtgcatgctaacaggtccgagtggacagatgaattaaacacgtacagagtggaagcagcttggaaattgtcacagtgggatttggtggaaaactatttggcagcagatggaaaatctacaacatggagtgtcagactgggacagctattattatcagccaaaaaaagagatatcacagctttttatgactcactgaaactagtgagagcagaacaaattgtacctctttcagctgcaagctttgaaagaggctcctaccaacgaggatatgaatatattgtgagattgcacatgttatgtgagttggagcatagcatcaaaccacttttccagcattctccaggtgacagttctcaagaagattctctaaactgggtagctcgactagaaatgacccagaattcctacagagccaaggagcctatcctggctctccggagggctttactaagcctcaacaaaagaccagattacaatgaaatggttggagaatgctggctgcagagtgccagggtagctagaaaggctggtcaccaccagacagcctacaatgctctccttaatgcaggggaatcacgactcgctgaactgtacgtggaaagggcaaagtggctctggtccaagggtgatgttcaccaggcactaattgttcttcaaaaaggtgttgaattatgttttcctgaaaatgaaaccccacctgagggtaagaacatgttaatccatggtcgagctatgctactagtgggccgatttatggaagaaacagctaactttgaaagcaatgcaattatgaaaaaatataaggatgtgaccgcgtgcctgccagaatgggaggatgggcatttttaccttgccaagtactatgacaaattgatgcccatggtcacagacaacaaaatggaaaagcaaggtgatctcatccggtatatagttcttcattttggcagatctctacaatatggaaatcagttcatatatcagtcaatgccacgaatgttaactctatggcttgattatggtacaaaggcatatgaatgggaaaaagctggccgctccgatcgtgtacaaatgaggaatgatttgggtaaaataaacaaggttatcacagagcatacaaactatttagctccatatcaatttttgactgctttttcacaattgatctctcgaatttgtcattctcacgatgaagtttttgttgtcttgatggaaataatagccaaagtatttctagcctatcctcaacaagcaatgtggatgatgacagctgtgtcaaagtcatcttatcccatgcgtgtgaacagatgcaaggaaatcctcaataaagctattcatatgaaaaaatccttagagaagtttgttggagatgcaactcgcctaacagataagcttctagaattgtgcaataaaccggttgatggaagtagttccacattaagcatgagcactcattttaaaatgcttaaaaagctggtagaagaagcaacatttagtgaaatcctcattcctctacaatcagtcatgatacctacacttccatcaattctgggtacccatgctaaccatgctagccatgaaccatttcctggacattgggcctatattgcagggtttgatgatatggtggaaattcttgcttctcttcagaaaccaaagaagatttctttaaaaggctcagatggaaagttctacatcatgatgtgtaagccaaaagatgacctgagaaaggattgtagactaatggaattcaattccttgattaataagtgcttaagaaaagatgcagagtctcgtagaagagaacttcatattcgaacatatgcagttattccactaaatgatgaatgtgggattattgaatgggtgaacaacactgctggtttgagacctattctgaccaaactatataaagaaaagggagtgtatatgacaggaaaagaacttcgccagtgtatgctaccaaagtcagcagctttatctgaaaaactcaaagtattccgagaatttctcctgcccaggcatcctcctatttttcatgagtggtttctgagaacattccctgatcctacatcatggtacagtagtagatcagcttactgccgttccactgcagtaatgtcaatggttggttatattctggggcttggagaccgtcatggtgaaaatattctctttgattctttgactggtgaatgcgtacatgtagatttcaattgtcttttcaataagggagaaacctttgaagttccagaaattgtgccatttcgcctgactcataatatggttaatggaatgggtcctatgggaacagagggtctttttcgaagagcatgtgaagttacaatgaggctgatgcgtgatcagcgagagcctttaatgagtgtcttaaagacttttctacatgatcctcttgtggaatggagtaaaccagtgaaagggcattccaaagcgccactgaatgaaactggagaagttgtcaatgaaaaggccaagacccatgttcttgacattgagcagcgactacaaggtgtaatcaagactcgaaatagagtgacaggactgccgttatctattgaaggacatgtgcattaccttatacaagaagctactgatgaaaacttactatgccagatgtatcttggttggactccatatatgtgaaatgaaattatgtaaaagaatatgttaataatctaaaagtaatgcatttggtatgaatctgtggttgtatctgttcaattctaaagtacaacataaatttacgttctcagcaactgttatttctctctgatcattaattatatgtaaaataatatacattcagttattaagaaataaactgctttcttaataaaaaaaaaaaaaaaaaaaaaaaaaaaaaaaaaa

SEQ ID NO: 5 - DNA, 13147 bp, Homo sapiens
ccggagcccgagccgaagggcgagccgcaaacgctaagtcgctggccattggtggacatggcgcaggcgcgtttgctccgacgggccgaatgttttggggcagtgttttgagcgcggagaccgcgtgatactggatgcgcatgggcataccgtgctctgcggctgcttggcgttgcttcttcctccagaagtgggcgctgggcagtcacgcagggtttgaaccggaagcgggagtaggtagctgcgtggctaacggagaaaagaagccgtggccgcgggaggaggcgagaggagtcgggatctgcgctgcagccaccgccgcggttgatactactttgaccttccgagtgcagtgacagtgatgtgtgttctgaaattgtgaaccatgagtctagtacttaatgatctgcttatctgctgccgtcaactagaacatgatagagctacagaacgaaagaaagaagttgagaaatttaagcgcctgattcgagatcctgaaacaattaaacatctagatcggcattcagattccaaacaaggaaaatatttgaattgggatgctgtttttagatttttacagaaatatattcagaaagaaacagaatgtctgagaatagcaaaaccaaatgtatcagcctcaacacaagcctccaggcagaaaaagatgcaggaaatcagtagtttggtcaaatacttcatcaaatgtgcaaacagaagagcacctaggctaaaatgtcaagaactcttaaattatatcatggatacagtgaaagattcatctaatggtgctatttacggagctgattgtagcaacatactactcaaagacattctttctgtgagaaaatactggtgtgaaatatctcagcaacagtggttagaattgttctctgtgtacttcaggctctatctgaaaccttcacaagatgttcatagagttttagtggctagaataattcatgctgttaccaaaggatgctgttctcagactgacggattaaattccaaatttttggactttttttccaaggctattcagtgtgcgagacaagaaaagagctcttcaggtctaaatcatatcttagcagctcttactatcttcctcaagactttggctgtcaactttcgaattcgagtgtgtgaattaggagatgaaattcttcccactttgctttatatttggactcaacataggcttaatgattctttaaaagaagtcattattgaattatttcaactgcaaatttatatccatcatccgaaaggagccaaaacccaagaaaaaggtgcttatgaatcaacaaaatggagaagtattttatacaacttatatgatctgctagtgaatgagataagtcatataggaagtagaggaaagtattcttcaggatttcgtaatattgccgtcaaagaaaatttgattgaattgatggcagatatctgtcaccaggtttttaatgaagataccagatccttggagatttctcaatcttacactactacacaaagagaatctagtgattacagtgtcccttgcaaaaggaagaaaatagaactaggctgggaagtaataaaagatcaccttcagaagtcacagaatgattttgatcttgtgccttggctacagattgcaacccaattaatatcaaagtatcctgcaagtttacctaactgtgagctgtctccattactgatgatactatctcagcttctaccccaacagcgacatggggaacgtacaccatatgtgttacgatgccttacggaagttgcattgtgtcaagacaagaggtcaaacctagaaagctcacaaaagtcagatttattaaaactctggaataaaatttggtgtattacctttcgtggtataagttctgagcaaatacaagctgaaaactttggcttacttggagccataattcagggtagtttagttgaggttgacagagaattctggaagttatttactgggtcagcctgcagaccttcatgtcctgcagtatgctgtttgactttggcactgaccaccagtatagttccaggaacggtaaaaatgggaatagagcaaaatatgtgtgaagtaaatagaagcttttctttaaaggaatcaataatgaaatggctcttattctatcagttagagggtgacttagaaaatagcacagaagtgcctccaattcttcacagtaattttcctcatcttgtactggagaaaattcttgtgagtctcactatgaaaaactgtaaagctgcaatgaattttttccaaagcgtgccagaatgtgaacaccaccaaaaagataaagaagaactttcattctcagaagtagaagaactatttcttcagacaacttttgacaagatggactttttaaccattgtgagagaatgtggtatagaaaagcaccagtccagtattggcttctctgtccaccagaatctcaaggaatcactggatcgctgtcttctgggattatcagaacagcttctgaataattactcatctgagattacaaattcagaaactcttgtccggtgttcacgtcttttggtgggtgtccttggctgctactgttacatgggtgtaatagctgaagaggaagcatataagtcagaattattccagaaagccaagtctctaatgcaatgtgcaggagaaagtatcactctgtttaaaaataagacaaatgaggaattcagaattggttccttgagaaatatgatgcagctatgtacacgttgcttgagcaactgtaccaagaagagtccaaataagattgcatctggctttttcctgcgattgttaacatcaaagctaatgaatgacattgcagatatttgtaaaagtttagcatccttcatcaaaaagccatttgaccgtggagaagtagaatcaatggaagatgatactaatggaaatctaatggaggtggaggatcagtcatccatgaatctatttaacgattaccctgatagtagtgttagtgatgcaaacgaacctggagagagccaaagtaccataggtgccattaatcctttagctgaagaatatctgtcaaagcaagatctacttttcttagacatgctcaagttcttgtgtttgtgtgtaactactgctcagaccaatactgtgtcctttagggcagctgatattcggaggaaattgttaatgttaattgattctagcacgctagaacctaccaaatccctccacctgcatatgtatctaatgcttttaaaggagcttcctggagaagagtaccccttgccaatggaagatgttcttgaacttctgaaaccactatccaatgtgtgttctttgtatcgtcgtgaccaagatgtttgtaaaactattttaaaccatgtccttcatgtagtgaaaaacctaggtcaaagcaatatggactctgagaacacaagggatgctcaaggacagtttcttacagtaattggagcattttggcatctaacaaaggagaggaaatatatattctctgtaagaatggccctagtaaattgccttaaaactttgcttgaggctgatccttattcaaaatgggccattcttaatgtaatgggaaaagactttcctgtaaatgaagtatttacacaatttcttgctgacaatcatcaccaagttcgcatgttggctgcagagtcaatcaatagattgttccaggacacgaagggagattcttccaggttactgaaagcacttcctttgaagcttcagcaaacagcttttgaaaatgcatacttgaaagctcaggaaggaatgagagaaatgtcccatagtgctgagaaccctgaaactttggatgaaatttataatagaaaatctgttttactgacgttgatagctgtggttttatcctgtagccctatctgcgaaaaacaggctttgtttgccctgtgtaaatctgtgaaagagaatggattagaacctcaccttgtgaaaaaggttttagagaaagtttctgaaacttttggatatagacgtttagaagactttatggcatctcatttagattatctggttttggaatggctaaatcttcaagatactgaatacaacttatcttcttttccttttattttattaaactacacaaatattgaggatttctatagatcttgttataaggttttgattccacatctggtgattagaagtcattttgatgaggtgaagtccattgctaatcagattcaagaggactggaaaagtcttctaacagactgctttccaaagattcttgtaaatattcttccttattttgcctatgagggtaccagagacagtgggatggcacagcaaagagagactgctaccaaggtctatgatatgcttaaaagtgaaaacttattgggaaaacagattgatcacttattcattagtaatttaccagagattgtggtggagttattgatgacgttacatgagccagcaaattctagtgccagtcagagcactgacctctgtgacttttcaggggatttggatcctgctcctaatccacctcattttccatcgcatgtgattaaagcaacatttgcctatatcagcaattgtcataaaaccaagttaaaaagcattttagaaattctttccaaaagccctgattcctatcagaaaattcttcttgccatatgtgagcaagcagctgaaacaaataatgtttataagaagcacagaattcttaaaatatatcacctgtttgttagtttattactgaaagatataaaaagtggcttaggaggagcttgggcctttgttcttcgagacgttatttatactttgattcactatatcaaccaaaggccttcttgtatcatggatgtgtcattacgtagcttctccctttgttgtgacttattaagtcaggtttgccagacagccgtgacttactgtaaggatgctctagaaaaccatcttcatgttattgttggtacacttataccccttgtgtatgagcaggtggaggttcagaaacaggtattggacttgttgaaatacttagtgatagataacaaggataatgaaaacctctatatcacgattaagcttttagatccttttcctgaccatgttgtttttaaggatttgcgtattactcagcaaaaaatcaaatacagtagaggacccttttcactcttggaggaaattaaccattttctctcagtaagtgtttatgatgcacttccattgacaagacttgaaggactaaaggatcttcgaagacaactggaactacataaagatcagatggtggacattatgagagcttctcaggataatccgcaagatgggattatggtgaaactagttgtcaatttgttgcagttatccaagatggcaataaaccacactggtgaaaaagaagttctagaggctgttggaagctgcttgggagaagtgggtcctatagatttctctaccatagctatacaacatagtaaagatgcatcttataccaaggcccttaagttatttgaagataaagaacttcagtggaccttcataatgctgacctacctgaataacacactggtagaagattgtgtcaaagttcgatcagcagctgttacctgtttgaaaaacattttagccacaaagactggacatagtttctgggagatttataagatgacaacagatccaatgctggcctatctacagccttttagaacatcaagaaaaaagtttttagaagtacccagatttgacaaagaaaacccttttgaaggcctggatgatataaatctgtggattcctctaagtgaaaatcatgacatttggataaagacactgacttgtgcttttttggacagtggaggcacaaaatgtgaaattcttcaattattaaagccaatgtgtgaagtgaaaactgacttttgtcagactgtacttccatacttgattcatgatattttactccaagatacaaatgaatcatggagaaatctgctttctacacatgttcagggatttttcaccagctgtcttcgacacttctcgcaaacgagccgatccacaacccctgcaaacttggattcagagtcagagcactttttccgatgctgtttggataaaaaatcacaaagaacaatgcttgctgttgtggactacatgagaagacaaaagagaccttcttcaggaacaatttttaatgatgctttctggctggatttaaattatctagaagttgccaaggtagctcagtcttgtgctgctcactttacagctttactctatgcagaaatctatgcagataagaaaagtatggatgatcaagagaaaagaagtcttgcatttgaagaaggaagccagagtacaactatttctagcttgagtgaaaaaagtaaagaagaaactggaataagtttacaggatcttctcttagaaatctacagaagtataggggagccagatagtttgtatggctgtggtggagggaagatgttacaacccattactagactacgaacatatgaacacgaagcaatgtggggcaaagccctagtaacatatgacctcgaaacagcaatcccctcatcaacacgccaggcaggaatcattcaggccttgcagaatttgggactctgccatattctttccgtctatttaaaaggattggattatgaaaataaagactggtgtcctgaactagaagaacttcattaccaagcagcatggaggaatatgcagtgggaccattgcacttccgtcagcaaagaagtagaaggaaccagttaccatgaatcattgtacaatgctctacaatctctaagagacagagaattctctacattttatgaaagtctcaaatatgccagagtaaaagaagtggaagagatgtgtaagcgcagccttgagtctgtgtattcgctctatcccacacttagcaggttgcaggccattggagagctggaaagcattggggagcttttctcaagatcagtcacacatagacaactctctgaagtatatattaagtggcagaaacactcccagcttctcaaggacagtgattttagttttcaggagcctatcatggctctacgcacagtcattttggagatcctgatggaaaaggaaatggacaactcacaaagagaatgtattaaggacattctcaccaaacaccttgtagaactctctatactggccagaactttcaagaacactcagctccctgaaagggcaatatttcaaattaaacagtacaattcagttagctgtggagtctctgagtggcagctggaagaagcacaagtattctgggcaaaaaaggagcagagtcttgccctgagtattctcaagcaaatgatcaagaagttggatgccagctgtgcagcgaacaatcccagcctaaaacttacatacacagaatgtctgagggtttgtggcaactggttagcagaaacgtgcttagaaaatcctgcggtcatcatgcagacctatctagaaaaggcagtagaagttgctggaaattatgatggagaaagtagtgatgagctaagaaatggaaaaatgaaggcatttctctcattagcccggttttcagatactcaataccaaagaattgaaaactacatgaaatcatcggaatttgaaaacaagcaagctctcctgaaaagagccaaagaggaagtaggtctccttagggaacataaaattcagacaaacagatacacagtaaaggttcagcgagagctggagttggatgaattagccctgcgtgcactgaaagaggatcgtaaacgcttcttatgtaaagcagttgaaaattatatcaactgcttattaagtggagaagaacatgatatgtgggtattccgactttgttccctctggcttgaaaattctggagtttctgaagtcaatggcatgatgaagagagacggaatgaagattccaacatataaatttttgcctcttatgtaccaattggctgctagaatggggaccaagatgatgggaggcctaggatttcatgaagtcctcaataatctaatctctagaatttcaatggatcacccccatcacactttgtttattatactggccttagcaaatgcaaacagagatgaatttctgactaaaccagaggtagccagaagaagcagaataactaaaaatgtgcctaaacaaagctctcagcttgatgaggatcgaacagaggctgcaaatagaataatatgtactatcagaagtaggagacctcagatggtcagaagtgttgaggcactttgtgatgcttatattatattagcaaacttagatgccactcagtggaagactcagagaaaaggcataaatattccagcagaccagccaattactaaacttaagaatttagaagatgttgttgtccctactatggaaattaaggtggaccacacaggagaatatggaaatctggtgactatacagtcatttaaagcagaatttcgcttagcaggaggtgtaaatttaccaaaaataatagattgtgtaggttccgatggcaaggagaggagacagcttgttaagggccgtgatgacctgagacaagatgctgtcatgcaacaggtcttccagatgtgtaatacattactgcagagaaacacggaaactaggaagaggaaattaactatctgtacttataaggtggttcccctctctcagcgaagtggtgttcttgaatggtgcacaggaactgtccccattggtgaatttcttgttaacaatgaagatggtgctcataaaagatacaggccaaatgatttcagtgcctttcagtgccaaaagaaaatgatggaggtgcaaaaaaagtcttttgaagagaaatatgaagtcttcatggatgtttgccaaaattttcaaccagttttccgttacttctgcatggaaaaattcttggatccagctatttggtttgagaagcgattggcttatacgcgcagtgtagctacttcttctattgttggttacatacttggacttggtgatagacatgtacagaatatcttgataaatgagcagtcagcagaacttgtacatatagatctaggtgttgcttttgaacagggcaaaatccttcctactcctgagacagttccttttagactcaccagagatattgtggatggcatgggcattacgggtgttgaaggtgtcttcagaagatgctgtgagaaaaccatggaagtgatgagaaactctcaggaaactctgttaaccattgtagaggtccttctatatgatccactctttgactggaccatgaatcctttgaaagctttgtatttacagcagaggccggaagatgaaactgagcttcaccctactctgaatgcagatgaccaagaatgcaaacgaaatctcagtgatattgaccagagtttcaacaaagtagctgaacgtgtcttaatgagactacaagagaaactgaaaggagtggaagaaggcactgtgctcagtgttggtggacaagtgaatttgctcatacagcaggccatagaccccaaaaatctcagccgacttttcccaggatggaaagcttgggtgtgatcttcagtatatgaattaccctttcattcagcctttagaaattatattttagcctttatttttaacctgccaacatactttaagtagggattaatatttaagtgaactattgtgggtttttttgaatgttggttttaatacttgatttaatcaccactcaaaaatgttttgatggtcttaaggaacatctctgctttcactctttagaaataatggtcattcgggctgggcgcagcggctcacgcctgtaatcccagcactttgggaggccgaggtgagcggatcacaaggtcaggagttcgagaccagcctggccaagagaccagcctggccagtatggtgaaaccctgtctctactaaaaatacaaaaattagccgagcatggtggcgggcacctgtaatcccagctactcgagaggctgaggcaggagaatctcttgaacctgggaggtgaaggttgctgtgggccaaaatcatgccattgcactccagcctgggtgacaagagcgaaactccatctcaaaaaaaaaaaaaaaaaaacagaaacgtatttggatttttcctagtaagatcactcagtgttactaaataatgaagttgttatggagaacaaatttcaaagacacagttagtgtagttactatttttttaagtgtgtattaaaacttctcattctattctctttatcttttaagcccttctgtactgtccatgtatgttatctttctgtgataacttcatagattgccttctagttcatgaattctcttgtcagatgtatataatctcttttaccctatccattgggcttcttctttcagaaattgtttttcatttctaattatgcatcatttttcagatctctgtttcttgatgtcatttttaatgtttttttaatgttttttatgtcactaattattttaaatgtctgtacttgatagacactgtaatagttctattaaatttagttcctgctgtttatatctgttgatttttgtatttgataggctgttcatccagttttgtctttttgaaaagtgagtttattttcagcaaggctttatctatgggaatcttgagtgtctgtttatgtcatattcccagggctgttgctgcacacaagcccattcttattttaatttcttggctttagggtttccatacctgaagtgtagcataaatactgataggagatttcccaggccaaggcaaacacacttcctcctcatctccttgtgctagtgggcagaatatttgattgatgcctttttcactgagagtataagcttccatgtgtcccacctttatggcaggggtggaaggaggtacatttaattcccactgcctgcctttggcaagccctgggttctttgctccccatatagatgtctaagctaaaagccgtgggttaatgagactggcaaattgttccaggacagctacagcatcagctcacatattcacctctctggtttttcattcccctcatttttttctgagacagagtcttgctctgtcacccaggctggagtgcagtggcatgatctcagctcactgaaacctctgcctcctgggttcaagcaattctcctgcctcagcctcccgagtagctgggactacaggcgtgtgccaacacgcccggctaattttttgtatttttattagagacggagtttcaccgtgttagccaggatggtctcgatcgcttgacctcgtgatccaccctcctcggcctcccaaagtgctgggattacaggtgtgagccaccgcgcccggcctcattcccctcatttttgaccgtaaggatttcccctttcttgtaagttctgctatgtatttaaaagaatgttttctacattttatccagcatttctctgtgttctgttggaagggaagggcttaggtatctagtttgatacataggtagaagtggaacatttctctgtcccccagctgtcatcatataagataaacatcagataaaaagccacctgaaagtaaaactactgactcgtgtattagtgagtataatctcttctccatccttaggaaaatgttcatcccagctgcggagattaacaaatgggtgattgagctttctcctcgtatttggaccttgaaggttatataaatttttttcttatgaagagttggcatttctttttattgccaatggcaggcactcattcatatttgatctcctcaccttcccctcccctaaaaccaatctccagaactttttggactataaatttcttggtttgacttctggagaactgttcagaatattactttgcatttcaaattacaaacttaccttggtgtatctttttcttacaagctgcctaaatgaatatttggtatatattggtagttttattactatagtaaatcaaggaaatgcagtaaacttaaaatgtctttaagaaagccctgaaatcttcatgggtgaaattagaaattatcaactagataatagtatagataaatgaatttgtagctaattcttgctagttgttgcatccagagagctttgaataacatcattaatctactctttagccttgcatggtatgctatgaggctcctgttctgttcaagtattctaatcaatggctttgaaaagtttatcaaatttacatacagatcacaagcctaggagaaataactaattcacagatgacagaattaagattataaaagattttttttttgtaattttagtagagacagggttgccattgtattccagccttggcgacagagcaagactctgcctcaaaaaaaaaaaaaaaaaggttttggcaagctggaactctttctgcaaatgactaagatagaaaactgccaaggacaaatgaggagtagttagattttgaaaatattaatcatagaatagttgttgtatgctaagtcactgacccatattatgtacagcatttctgatctttactttgcaagattagtgatactatcccaatacactgctggagaaatcagaatttggagaaataagttgtccaaggcaagaagatagtaaattataagtacaagtgtaatatggacagtatctaacttgaaaagatttcaggcgaaaagaatctggggtttgccagtcagttgctcaaaaggtcaatgaaaaccaaatagtgaagctatcagagaagctaataaattatagactgcttgaacagttgtgtccagattaagggagataatagctttcccaccctactttgtgcaggtcatacctccccaaagtgtttacctaatcagtaggttcacaaactcttggtcattatagtatatgcctaaaatgtatgcacttaggaatgctaaaaatttaaatatggtctaaagcaaataaaagcaaagaggaaaaactttggacagcgtaaagactagaatagtcttttaaaaagaaagccagtatattggtttgaaatatagagatgtgtcccaatttcaagtattttaattgcaccttaatgaaattatctattttctatagattttagtactattgaatgtattactttactgttacctgaatttattataaagtgtttttgaataaataattctaaaagc

SEQ ID NO: 6 - protein, 2644 aa, Homo sapiens
MGEHGLELASMIPALRELGSATPEEYNTVVQKPRQILCQFIDRILTDVNVVAVELVKKTDSQPTSVMLLDFIQHIMKSSPLMFVNVSGSHERKGSCIEFSNWIITRLLRIAATPSCHLLHKKICEVICSLLFLFKSKSPAIFGVLTKELLQLFEDLVYLHRRNVMGHAVEWPVVMSRFLSQLDEHMGYLQSAPLQLMSMQNLEFIEVTLLMVLTRIIAIVFFRRQELLLWQIGCVLLEYGSPKIKSLAISFLTELFQLGGLPAQPASTFFSSFLELLKHLVEMDTDQLKLYEEPLSKLIKTLFPFEAEAYRNIEPVYLNMLLEKLCVMFEDGVLMRLKSDLLKAALCHLLQYFLKFVPAGYESALQVRKVYVRNICKALLDVLGIEVDAEYLLGPLYAALKMESMEIIEEIQCQTQQENLSSNSDGISPKRRRLSSSLNPSKRAPKQTEEIKHVDMNQKSILWSALKQKAESLQISLEYSGLKNPVIEMLEGIAVVLQLTALCTVHCSHQNMNCRTFKDCQHKSKKKPSVVITWMSLDFYTKVLKSCRSLLESVQKLDLEATIDKVVKIYDALIYMQVNSSFEDHILEDLCGMLSLPWIYSHSDDGCLKLTTFAANLLTLSCRISDSYSPQAQSRCVFLLTLFPRRIFLEWRTAVYNWALQSSHEVIRASCVSGFFILLQQQNSCNRVPKILIDKVKDDSDIVKKEFASILGQLVCTLHGMFYLTSSLTEPFSEHGHVDLFCRNLKATSQHECSSSQLKASVCKPFLFLLKKKIPSPVKLAFIDNLHHLCKHLDFREDETDVKAVLGTLLNLMEDPDKDVRVAFSGNIKHILESLDSEDGFIKELFVLRMKEAYTHAQISRNNELKDTLILTTGDIGRAAKGDLVPFALLHLLHCLLSKSASVSGAAYTEIRALVAAKSVKLQSFFSQYKKPICQFLVESLHSSQMTALPNTPCQNADVRKQDVAHQREMALNTLSEIANVFDFPDLNRFLTRTLQVLLPDLAAKASPAASALIRTLGKQLNVNRREILINNFKYIFSHLVCSCSKDELERALHYLKNETEIELGSLLRQDFQGLHNELLLRIGEHYQQVFNGLSILASFASSDDPYQGPRDIISPELMADYLQPKLLGILAFFNMQLLSSSVGIEDKKMALNSLMSLMKLMGPKHVSSVRVKMMTTLRTGLRFKDDFPELCCRAWDCFVRCLDHACLGSLLSHVIVALLPLIHIQPKETAAIFHYLIIENRDAVQDFLHEIYFLPDHPELKKIKAVLQEYRKETSESTDLQTTLQLSMKAIQHENVDVRIHALTSLKETLYKNQEKLIKYATDSETVEPIISQLVTVLLKGCQDANSQARLLCGECLGELGAIDPGRLDFSTTETQGKDFTFVTGVEDSSFAYGLLMELTRAYLAYADNSRAQDSAAYAIQELLSIYDCREMETNGPGHQLWRRFPEHVREILEPHLNTRYKSSQKSTDWSGVKKPIYLSKLGSNFAEWSASWAGYLITKVRHDLASKIFTCCSIMMKHDFKVTIYLLPHILVYVLLGCNQEDQQEVYAEIMAVLKHDDQHTINTQDIASDLCQLSTQTVFSMLDHLTQWARHKFQALKAEKCPHSKSNRNKVDSMVSTVDYEDYQSVTRFLDLIPQDTLAVASFRSKAYTRAVMHFESFITEKKQNIQEHLGFLQKLYAAMHEPDGVAGVSAIRKAEPSLKEQILEHESLGLLRDATACYDRAIQLEPDQIIHYHGVVKSMLGLGQLSTVITQVNGVHANRSEWTDELNTYRVEAAWKLSQWDLVENYLAADGKSTTWSVRLGQLLLSAKKRDITAFYDSLKLVRAEQIVPLSAASFERGSYQRGYEYIVRLHMLCELEHSIKPLFQHSPGDSSQEDSLNWVARLEMTQNSYRAKEPILALRRALLSLNKRPDYNEMVGECWLQSARVARKAGHHQTAYNALLNAGESRLAELYVERAKWLWSKGDVHQALIVLQKGVELCFPENETPPEGKNMLIHGRAMLLVGRFMEETANFESNAIMKKYKDVTACLPEWEDGHFYLAKYYDKLMPMVTDNKMEKQGDLIRYIVLHFGRSLQYGNQFIYQSMPRMLTLWLDYGTKAYEWEKAGRSDRVQMRNDLGKINKVITEHTNYLAPYQFLTAFSQLISRICHSHDEVFVVLMEIIAKVFLAYPQQAMWMMTAVSKSSYPMRVNRCKEILNKAIHMKKSLEKFVGDATRLTDKLLELCNKPVDGSSSTLSMSTHFKMLKKLVEEATFSEILIPLQSVMIPTLPSILGTHANHASHEPFPGHWAYIAGFDDMVEILASLQKPKKISLKGSDGKFYIMMCKPKDDLRKDCRLMEFNSLINKCLRKDAESRRRELHIRTYAVIPLNDECGIIEWVNNTAGLRPILTKLYKEKGVYMTGKELRQCMLPKSAALSEKLKVFREFLLPRHPPIFHEWFLRTFPDPTSWYSSRSAYCRSTAVMSMVGYILGLGDRHGENILFDSLTGECVHVDFNCLFNKGETFEVPEIVPFRLTHNMVNGMGPMGTEGLFRRACEVTMRLMRDQREPLMSVLKTFLHDPLVEWSKPVKGHSKAPLNETGEVVNEKAKTHVLDIEQRLQGVIKTRNRVTGLPLSIEGHVHYLIQEATDENLLCQMYLGWTPYM

SEQ ID NO: 7 - protein, 3056 aa, Homo sapiens
MSLVLNDLLICCRQLEHDRATERKKEVEKFKRLIRDPETIKHLDRHSDSKQGKYLNWDAVFRFLQKYIQKETECLRIAKPNVSASTQASRQKKMQEISSLVKYFIKCANRRAPRLKCQELLNYIMDTVKDSSNGAIYGADCSNILLKDILSVRKYWCEISQQQWLELFSVYFRLYLKPSQDVHRVLVARIIHAVTKGCCSQTDGLNSKFLDFFSKAIQCARQEKSSSGLNHILAALTIFLKTLAVNFRIRVCELGDEILPTLLYIWTQHRLNDSLKEVIIELFQLQIYIHHPKGAKTQEKGAYESTKWRSILYNLYDLLVNEISHIGSRGKYSSGFRNIAVKENLIELMADICHQVFNEDTRSLEISQSYTTTQRESSDYSVPCKRKKIELGWEVIKDHLQKSQNDFDLVPWLQIATQLISKYPASLPNCELSPLLMILSQLLPQQRHGERTPYVLRCLTEVALCQDKRSNLESSQKSDLLKLWNKIWCITFRGISSEQIQAENFGLLGAIIQGSLVEVDREFWKLFTGSACRPSCPAVCCLTLALTTSIVPGTVKMGIEQNMCEVNRSFSLKESIMKWLLFYQLEGDLENSTEVPPILHSNFPHLVLEKILVSLTMKNCKAAMNFFQSVPECEHHQKDKEELSFSEVEELFLQTTFDKMDFLTIVRECGIEKHQSSIGFSVHQNLKESLDRCLLGLSEQLLNNYSSEITNSETLVRCSRLLVGVLGCYCYMGVIAEEEAYKSELFQKAKSLMQCAGESITLFKNKTNEEFRIGSLRNMMQLCTRCLSNCTKKSPNKIASGFFLRLLTSKLMNDIADICKSLASFIKKPFDRGEVESMEDDTNGNLMEVEDQSSMNLFNDYPDSSVSDANEPGESQSTIGAINPLAEEYLSKQDLLFLDMLKFLCLCVTTAQTNTVSFRAADIRRKLLMLIDSSTLEPTKSLHLHMYLMLLKELPGEEYPLPMEDVLELLKPLSNVCSLYRRDQDVCKTILNHVLHVVKNLGQSNMDSENTRDAQGQFLTVIGAFWHLTKERKYIFSVRMALVNCLKTLLEADPYSKWAILNVMGKDFPVNEVFTQFLADNHHQVRMLAAESINRLFQDTKGDSSRLLKALPLKLQQTAFENAYLKAQEGMREMSHSAENPETLDEIYNRKSVLLTLIAVVLSCSPICEKQALFALCKSVKENGLEPHLVKKVLEKVSETFGYRRLEDFMASHLDYLVLEWLNLQDTEYNLSSFPFILLNYTNIEDFYRSCYKVLIPHLVIRSHFDEVKSIANQIQEDWKSLLTDCFPKILVNILPYFAYEGTRDSGMAQQRETATKVYDMLKSENLLGKQIDHLFISNLPEIVVELLMTLHEPANSSASQSTDLCDFSGDLDPAPNPPHFPSHVIKATFAYISNCHKTKLKSILEILSKSPDSYQKILLAICEQAAETNNVYKKHRILKIYHLFVSLLLKDIKSGLGGAWAFVLRDVIYTLIHYINQRPSCIMDVSLRSFSLCCDLLSQVCQTAVTYCKDALENHLHVIVGTLIPLVYEQVEVQKQVLDLLKYLVIDNKDNENLYITIKLLDPFPDHVVFKDLRITQQKIKYSRGPFSLLEEINHFLSVSVYDALPLTRLEGLKDLRRQLELHKDQMVDIMRASQDNPQDGIMVKLVVNLLQLSKMAINHTGEKEVLEAVGSCLGEVGPIDFSTIAIQHSKDASYTKALKLFEDKELQWTFIMLTYLNNTLVEDCVKVRSAAVTCLKNILATKTGHSFWEIYKMTTDPMLAYLQPFRTSRKKFLEVPRFDKENPFEGLDDINLWIPLSENHDIWIKTLTCAFLDSGGTKCEILQLLKPMCEVKTDFCQTVLPYLIHDILLQDTNESWRNLLSTHVQGFFTSCLRHFSQTSRSTTPANLDSESEHFFRCCLDKKSQRTMLAVVDYMRRQKRPSSGTIFNDAFWLDLNYLEVAKVAQSCAAHFTALLYAEIYADKKSMDDQEKRSLAFEEGSQSTTISSLSEKSKEETGISLQDLLLEIYRSIGEPDSLYGCGGGKMLQPITRLRTYEHEAMWGKALVTYDLETAIPSSTRQAGIIQALQNLGLCHILSVYLKGLDYENKDWCPELEELHYQAAWRNMQWDHCTSVSKEVEGTSYHESLYNALQSLRDREFSTFYESLKYARVKEVEEMCKRSLESVYSLYPTLSRLQAIGELESIGELFSRSVTHRQLSEVYIKWQKHSQLLKDSDFSFQEPIMALRTVILEILMEKEMDNSQRECIKDILTKHLVELSILARTFKNTQLPERAIFQIKQYNSVSCGVSEWQLEEAQVFWAKKEQSLALSILKQMIKKLDASCAANNPSLKLTYTECLRVCGNWLAETCLENPAVIMQTYLEKAVEVAGNYDGESSDELRNGKMKAFLSLARFSDTQYQRIENYMKSSEFENKQALLKRAKEEVGLLREHKIQTNRYTVKVQRELELDELALRALKEDRKRFLCKAVENYINCLLSGEEHDMWVFRLCSLWLENSGVSEVNGMMKRDGMKIPTYKFLPLMYQLAARMGTKMMGGLGFHEVLNNLISRISMDHPHHTLFIILALANANRDEFLTKPEVARRSRITKNVPKQSSQLDEDRTEAANRIICTIRSRRPQMVRSVEALCDAYIILANLDATQWKTQRKGINIPADQPITKLKNLEDVVVPTMEIKVDHTGEYGNLVTIQSFKAEFRLAGGVNLPKIIDCVGSDGKERRQLVKGRDDLRQDAVMQQVFQMCNTLLQRNTETRKRKLTICTYKVVPLSQRSGVLEWCTGTVPIGEFLVNNEDGAHKRYRPNDFSAFQCQKKMMEVQKKSFEEKYEVFMDVCQNFQPVFRYFCMEKFLDPAIWFEKRLAYTRSVATSSIVGYILGLGDRHVQNILINEQSAELVHIDLGVAFEQGKILPTPETVPFRLTRDIVDGMGITGVEGVFRRCCEKTMEVMRNSQETLLTIVEVLLYDPLFDWTMNPLKALYLQQRPEDETELHPTLNADDQECKRNLSDIDQSFNKVAERVLMRLQEKLKGVEEGTVLSVGGQVNLLIQQAIDPKNLSRLFPGWKAWV